(19) United States
(12) Patent Application Publication
(10) Pub. No.: US 2024/0151702 A1
(43) Pub. Date: May 9, 2024

(54) METHODS OF MAKING AND ASSESSING ALLERGENIC TREE NUT COMPOSITIONS

(71) Applicant: SOCIETE DES PRODUITS NESTLE S.A., Vevey (CH)

(72) Inventors: JOUMANA ZEID, Brisbane, CA (US); LYNN GENNARO, Brisbane, CA (US); CHRISTINA C. CAPULE, Brisbane, CA (US); NEERAJ R. PAKALA, Brisbane, CA (US)

(21) Appl. No.: 18/548,674

(22) PCT Filed: Mar. 4, 2022
(86) PCT No.: PCT/IB2022/051958

§ 371 Date: Sep. 1, 2023

Related U.S. Application Data
(60) Provisional application No. 63/157,558, filed on Mar. 5, 2021.

(51) Int. Cl. G01N 33/15; G01N 30/88

(52) U.S. Cl. CPC G01N 33/15; G01N 30/88; G01N 2030/027

(57) ABSTRACT

The present disclosure relates to methods of assessing mixed tree nut powder compositions for pharmaceutical suitability, methods of manufacturing pharmaceutically suitable mixed tree nut powder compositions, and methods of manufacturing pharmaceutical powder compositions comprising at least a first type of tree nut flour.

### CROSS-REFERENCE TO RELATED APPLICATIONS

This application claims priority benefit to U.S. Provisional Application No. 63/157,558, filed on Mar. 5, 2021, entitled “METHODS OF MAKING AND ASSESSING ALLERGENIC TREE NUT COMPOSITIONS”, the contents of which are incorporated herein by reference for all purposes.

### FIELD OF THE INVENTION

The present disclosure relates to methods of assessing and/or making mixed tree nut powder compositions comprising tree nut flours, methods of manufacturing pharmaceutically suitable mixed tree nut powder compositions comprising tree nut flours, and methods of manufacturing pharmaceutical powder compositions comprising one or more different types of tree nut flour

### BACKGROUND OF THE INVENTION

Food allergy is an adverse reaction to food that is triggered by the immune system. Allergies to the nuts or drupes of trees (i.e., tree nut allergies) are often associated with severe, potentially life-threatening allergic reactions. Tree nut allergy can be lifelong and has been increasing in prevalence in recent years. Individuals with tree nut allergy are commonly advised to practice strict avoidance diets, which is the current standard of care, but accidental ingestion of tree nuts is common even with a strict avoidance diet. Strict adherence to an avoidance diet can be complicated by difficulty in interpreting food labels, the presence of undeclared or hidden allergens in commercially prepared foods, and inattention to or mistrust of food warning labels. Blom et al. followed a cohort of 157 food-allergic patients for a year and reported 151 allergic reactions, from 73 patients, associated with accidental food allergen exposures (Blom et al., Accidental food allergy reactions: Products and undeclared ingredients, J. Allergy Clin. Immunol., vol. 142, no. 3, pp. 865-875 (2018). Patient-provided food products were analyzed for allergenic content and allergens from more than one species of tree nut were detected, even though the allergens were not declared on the ingredient list. In pediatric patients reporting a tree nut allergy, almost 25% reported one or more food allergy related emergency room visit in the past year (see Gupta et al., The Public Health Impact of Parent-Reported Childhood Food Allergies in the United States, Pediatrics, vol. 142, no. 6, e20181235 (2018)). Thus, accidental exposure to tree nut is not uncommon, and is difficult to avoid.

Oral immunotherapy (OIT) is an emerging treatment option for food allergies including tree nut allergy. OIT of tree nut allergy involves progressive desensitization of individuals to the etiological agent of a particular tree nut allergy (for example, progressive exposure to walnuts in individuals with walnut allergy). Progressive desensitization is achieved by first administering doses of allergenic material below the dose necessary to trigger to an unwanted adverse allergic reaction (the reactive threshold), but high enough to induce a desensitization response. Over the course of the OIT, individuals can become progressively desensitized, whereby the reactive threshold to an allergen is continually increased. A goal of OIT to tree nuts is to desensitize individuals to a degree that they are able to tolerate accidental exposure to the tree nut. Additionally, through consistent OIT dosing, it may also be possible to achieve a state of sustained unresponsiveness, which is a state of persistent unresponsiveness to the tree nut.

It is common for individuals to present with allergic reactivity to multiple species of tree nuts. This can be due to cross-reactivity of homologous allergens or due to distinct allergies to two or more tree nut species. Because OIT regimens can last for months or even years, it is desirable to administer mixed allergen compositions containing allergens from two or more tree nut species. Thus, there is a need in the art for improved methods of assessing the pharmaceutical suitability of mixed tree nut powder compositions comprising tree nut flours for use in the OIT of one or more tree nut allergies. There is also a need in the art for methods of manufacturing pharmaceutically suitable powder compositions comprising tree nut flour.

### SUMMARY OF THE INVENTION

Described herein is a method of assessing a mixed tree nut powder composition for pharmaceutical suitability, wherein the mixed tree nut powder composition comprises at least a first tree nut flour and a second tree nut flour, the method comprising assaying the mixed tree nut powder composition by liquid chromatography to determine a ratio for an amount of the first tree nut flour and an amount of the second tree nut flour relative to each other, and assessing whether the mixed tree nut powder composition is pharmaceutically suitable, wherein the mixed tree nut powder composition is pharmaceutically suitable only if the ratio is within a predetermined threshold. In some embodiments, the mixed tree nut powder composition further comprises at least a third tree nut flour, wherein assaying the mixed tree nut powder composition by liquid chromatography further comprises: determining a second ratio for the amount of the first tree nut flour and the third tree nut flour relative to each other; and determining a third ratio for the amount of the second tree nut flour and the third tree nut flour relative to each other; wherein the mixed tree nut powder composition is pharmaceutically suitable only if the second ratio is within a second predetermined threshold and the third ratio is within a third predetermined threshold. In some embodiments, the mixed tree nut powder composition is assessed as pharmaceutically suitable and the method further comprises packaging the mixed tree nut powder composition. In some embodiments, the mixed tree nut powder composition is packaged in a pharmaceutically acceptable dosage form. In some embodiments, the mixed tree nut powder composition is assessed as pharmaceutically unsuitable, wherein the mixed tree nut powder composition is pharmaceutically unsuitable if the ratio is not within a predetermined threshold, the method further comprising discarding the mixed tree nut powder composition. In some embodiments, the mixed tree nut powder composition is assessed as pharmaceutically unsuitable, wherein the mixed tree nut powder composition is pharmaceutically unsuitable if one or more of the ratio, the second ratio, or the third ratio are not within a predetermined threshold, the method further comprising discarding the mixed tree nut powder composition. In some embodiments, the mixed tree nut powder composition further comprises one or more pharmaceutically acceptable excipients.

Further described herein is a method of manufacturing a pharmaceutically suitable mixed tree nut powder composition, the method comprising: forming the mixed tree nut powder composition, comprising mixing at least a first tree nut flour and a second tree nut flour; assaying the mixed tree nut powder composition by liquid chromatography to determine a ratio for an amount of the first tree nut flour and an amount of the second tree nut flour relative to each other; assessing whether the mixed tree nut powder composition is pharmaceutically suitable, wherein the mixed tree nut powder composition is pharmaceutically suitable only if the ratio is within a predetermined threshold; packaging the mixed tree nut powder composition if assessed to be pharmaceutically suitable. In some embodiments, forming the mixed tree nut powder composition further comprises mixing a third tree nut flour with the first tree nut flour and the second tree nut flour; wherein assaying the mixed tree nut powder composition by liquid chromatography further comprises: determining a second ratio for the amount of the first tree nut flour and the third tree nut flour relative to each other; and determining a third ratio for the amount of the second tree nut flour and the third tree nut flour relative to each other; wherein the mixed tree nut powder composition is pharmaceutically suitable only if the second ratio is within a second predetermined threshold and the third ratio is within a third predetermined threshold. In some embodiments, the mixed tree nut powder composition is packaged in a pharmaceutically acceptable dosage form.

In some embodiments, the liquid chromatography is reversed-phase ultra-high-performance liquid chromatography (RP-UPLC). In some embodiments, the predetermined threshold is a range set between about 0.75 and about 1.25. In some embodiments, the second predetermined threshold is a range set between about 0.75 and about 1.25. In some embodiments, the third predetermined threshold is a range set between about 0.75 and about 1.25.

In some embodiments, forming the mixed tree nut powder composition comprises mixing with one or more pharmaceutically acceptable excipients. In some embodiments, forming the mixed tree nut powder composition comprises simultaneously mixing the first tree nut flour, the second tree nut flour, and the one or more pharmaceutically acceptable excipients. In some embodiments, the mixed tree nut powder composition comprises the third tree nut flour, wherein forming the mixed tree nut powder composition comprises simultaneously mixing the first tree nut flour, the second tree nut flour, the third tree nut flour, and the one or more pharmaceutically acceptable excipients. In some embodiments, forming the mixed tree nut powder composition comprises: mixing the first tree nut flour and the second tree nut flour to form a tree nut flour mixture; and mixing the tree nut flour mixture with the one or more pharmaceutically acceptable excipients. In some embodiments, forming the mixed tree nut powder composition comprises: mixing the first tree nut flour, the second tree nut flour, and the third tree nut flour to form a tree nut flour mixture; and mixing the tree nut flour mixture with the one or more pharmaceutically acceptable excipients. In some embodiments, forming the mixed tree nut powder composition comprises: mixing the first tree nut flour with a first amount of the one or more pharmaceutically acceptable excipients to form a first mixture; mixing the second tree nut flour with a second amount of the one or more pharmaceutically acceptable excipients to form a second mixture; mixing the first mixture and the second mixture. In some embodiments, forming the mixed tree nut powder composition comprises: mixing the first tree nut flour with a first amount of the one or more pharmaceutically acceptable excipients to form a first mixture; mixing the second tree nut flour with a second amount of the one or more pharmaceutically acceptable excipients to form a second mixture; mixing the third tree nut flour with a third amount of the one or more pharmaceutically acceptable excipients to form a third mixture; and mixing the first mixture, the second mixture, and the third mixture. In some embodiments, the method further comprises mixing the mixed tree nut powder composition with one or more pharmaceutically acceptable excipients after assaying the mixed tree nut powder composition. In some embodiments, the mixed tree nut powder composition is packaged after mixing the mixed tree nut powder composition with the one or more pharmaceutically acceptable excipients.

In some embodiments, assaying comprises measuring a plurality of peak areas of sufficiently resolved quantitative peaks. In some embodiments, at least one sufficiently resolved quantitative peak is measured for each of the first tree nut flour and the second tree nut flour. In some embodiments, at least one sufficiently resolved quantitative peak is measured for each of the first tree nut flour, the second tree nut flour, and the third tree nut flour. In some embodiments, the plurality of sufficiently resolved quantitative peaks comprise a peak corresponding to peak C of FIG. 2, wherein Peak C is associated with cashew flour. In some embodiments, the plurality of sufficiently resolved quantitative peaks comprise a peak corresponding to peak W of FIG. 2, wherein Peak W is associated with walnut. In some embodiments, the plurality of sufficiently resolved quantitative peaks comprise a peak corresponding to peak H of FIG. 2, wherein Peak H is associated with hazelnut. In some embodiments, the plurality of sufficiently resolved quantitative peaks comprise a peak corresponding to Jug r 1 of walnut. In some embodiments, the plurality of sufficiently resolved quantitative peaks comprise a peak corresponding to a 2S albumin of walnut. In some embodiments, the plurality of sufficiently resolved quantitative peaks comprise a peak corresponding to a 2S albumin of hazelnut. In some embodiments, the plurality of sufficiently resolved quantitative peaks comprise a peak corresponding to a 2S albumin of cashew. In some embodiments, the plurality of sufficiently resolved quantitative peaks comprise a peak corresponding to Jug r 2 of walnut. In some embodiments, the plurality of sufficiently resolved quantitative peaks comprise a peak corresponding to Cor a 9 of hazelnut. In some embodiments, the plurality of sufficiently resolved quantitative peaks comprise a peak corresponding to Ana o 2 of cashew. In some embodiments, the plurality of sufficiently resolved quantitative peaks comprise one or more peaks corresponding to one or more cashew proteins selected from the group consisting of a 11S globulin, a 7S globulin, a lipid transfer protein, a Bet v 1-like protein, a profilin protein, and an oleosin protein. In some embodiments, the plurality of sufficiently resolved quantitative peaks comprise one or more peaks corresponding to one or more hazelnut proteins selected from the group consisting of a 11S globulin, a 7S globulin, a lipid transfer protein, a Bet v 1-like protein, a profilin protein, and an oleosin protein. In some embodiments, the plurality of sufficiently resolved quantitative peaks comprise one or more peaks corresponding to one or more walnut proteins selected from the group consisting of a 11S globulin, a 7S globulin, a lipid transfer protein, a Bet v 1-like protein, a profilin protein, and an oleosin protein.

In some embodiments, the method further comprises forming the first tree nut flour or the second tree nut flour from tree nuts. In some embodiments, the method further comprises forming the first tree nut flour, the second tree nut flour, or the third tree nut flour from tree nuts. In some embodiments, forming comprises roasting tree nuts. In some embodiments, roasting tree nuts comprises roasting at a temperature between about 90° C. and about 140° C. In some embodiments, roasting tree nuts comprises roasting at a temperature between about 90° C. and about 130° C. In some embodiments, the roasting is for between about 15 minutes and about 90 minutes. In some embodiments, forming the first tree nut flour, the second tree nut flour, or the third tree nut flour comprises pasteurizing tree nuts. In some embodiments, forming the first tree nut flour, the second tree nut flour, or the third tree nut flour comprises grinding tree nuts to produce ground tree nuts. In some embodiments, forming the first tree nut flour, the second tree nut flour, or the third tree nut flour comprises defatting a portion of fat from the ground tree nuts. In some embodiments, defatting a portion of fat from the ground tree nuts comprises pressing the ground tree nuts.

In some embodiments, the first tree nut flour or the second tree nut flour is walnut flour. In some embodiments, the first tree nut flour or the second tree nut flour is hazelnut flour, and the first tree nut flour and the second tree nut flour are different. In some embodiments, the first tree nut flour or the second tree nut flour is cashew flour, and the first tree nut flour and the second tree nut flour are different. In some embodiments, the first tree nut flour, the second tree nut flour, or the third tree nut flour is walnut flour. In some embodiments, the first tree nut flour, the second tree nut flour, or the third tree nut flour is hazelnut flour, and the first tree nut flour, the second tree nut flour, and the third tree nut flour are each different. In some embodiments, the first tree nut flour, the second tree nut flour, or the third tree nut flour is cashew flour, and the first tree nut flour, the second tree nut flour, and the third tree nut flour are each different.

In some embodiments, the pharmaceutically suitable mixed tree nut powder composition comprises about 1% to about 13% w/w total tree nut flour. In some embodiments, the pharmaceutically suitable mixed tree nut powder composition comprises about 13% to about 21% w/w total tree nut flour. In some embodiments, the pharmaceutically suitable mixed tree nut powder composition comprises about 21% to about 37% w/w total tree nut flour. In some embodiments, the pharmaceutically suitable mixed tree nut powder composition comprises about 37% to about 50% w/w total tree nut flour.

In some embodiments, the method further comprises identifying one or more sufficiently resolved quantitative peaks for the first tree nut flour, the second tree nut flour, or the third tree nut flour.

Further described herein is a method of manufacturing a pharmaceutical powder composition comprising at least a first type of tree nut flour, the method comprising: roasting at least a first type of tree nut at a temperature between about 90° C. and about 140° C. to obtain a desired moisture content; forming the at least a first type of tree nut flour using the at least a first type of tree nut after roasting; forming the pharmaceutical powder composition by mixing the at least a first type of tree nut flour with one or more pharmaceutically acceptable excipients; and packaging the pharmaceutical powder composition. In some embodiments, roasting the at least a first type of tree nut is at a temperature between about 90° C. and about 130° C. In some embodiments, the pharmaceutical powder composition is packaged in a dosage form in an amount suitable for oral immunotherapy. In some embodiments, the pharmaceutical powder composition comprises a second type of tree nut flour, and forming the pharmaceutical powder composition comprises mixing the at least a first type of tree nut flour, the second type of tree nut flour, and the one or more pharmaceutically acceptable excipients. In some embodiments, the method comprises roasting a second type of tree nut at a temperature between about 90° C. and about 140° C. to obtain a desired moisture content; and forming the second type of tree nut flour using the second type of tree nut after roasting. In some embodiments, roasting the second type of tree nut is at a temperature between about 90° C. and about 130° C. In some embodiments, the pharmaceutical powder composition comprises a third type of tree nut flour, and forming the pharmaceutical powder composition comprises mixing the at least a first type of tree nut flour, the second type of tree nut flour, the third type of tree nut flour, and the one or more pharmaceutically acceptable excipients. In some embodiments, the method comprises roasting a third type of tree nut at a temperature between about 90° C. and about 140° C. to obtain a desired moisture content; and forming the third type of tree nut flour using the third type of tree nut after roasting. In some embodiments, roasting the third type of tree nut is at a temperature between about 90° C. and about 130° C.

In some embodiments, the method comprises removing a portion of fat from the at least a first type of tree nut, the second type of tree nut, or the third type of tree nut. In some embodiments, removing a portion of fat comprises pressing. In some embodiments, the method comprises mixing measured amounts of the at least a first type of tree nut flour, the second type of tree nut flour, and the third type of tree nut flour. In some embodiments, roasting is at a temperature between about 90° C. and about 130° C. is for about 15 minutes to about 90 minutes.

In some embodiments, the at least a first type of tree nut flour has a median particle size of less than about 100 μm. In some embodiments, the at least a first type of tree nut flour has a Dv(90) less than about 300 μm. In some embodiments, the second type of tree nut flour has a median particle size of less than about 100 μm. In some embodiments, the second type of tree nut flour has a Dv(90) less than about 300 μm. In some embodiments, the third type of tree nut flour has a median particle size of less than about 100 μm. In some embodiments, the third type of tree nut flour has a Dv(90) less than about 300 μm.

In some embodiments, the at least a first type of tree nut has a moisture content of between about 0.5% and about 5% after roasting. In some embodiments, the second type of tree nut has a moisture content of between about 0.5% and about 5% after roasting. In some embodiments, the third type of tree nut has a moisture content between about 0.5% and about 5% after roasting.

In some embodiments, roasting comprises mixing during roasting. In some embodiments, roasting is performed with a multistage continuous nut roaster. In some embodiments, the method comprises grinding the at least a first type of tree nut, the second type of tree nut, or the third type of tree nut. In some embodiments, the method comprises removing a portion of fat from the at least a first type of tree nut, the second type of tree nut, or the third type of tree nut. In some embodiments, removing a portion of fat comprises pressing into a pressed cake. In some embodiments, the method comprises pasteurizing the at least a first type of tree nut, the second type of tree nut, or the third type of tree nut.

In some embodiments, the at least a first type of tree nut flour is walnut flour. In some embodiments, the at least a first type of tree nut flour is hazelnut flour. In some embodiments, the at least a first type of tree nut flour is cashew flour. In some embodiments, the second type of tree nut flour is hazelnut flour. In some embodiments, the second type of tree nut flour is cashew flour. In some embodiments, the second type of tree nut flour is walnut flour. In some embodiments, the third type of tree nut flour is cashew flour. In some embodiments, the third type of tree nut flour is hazelnut flour. In some embodiments, the third type of tree nut flour is walnut flour.

In some embodiments, each of the at least a first type of tree nut flour, the second type of tree nut flour, and the third type of tree nut flour are within about 90% and about 110% of a target weight. In some embodiments, the mixed tree nut powder composition comprises about 1% to about 12% w/w total tree nut flour. In some embodiments, the mixed tree nut powder composition comprises about 13% to about 20% w/w total tree nut flour. In some embodiments, the mixed tree nut powder composition comprises about 21% to about 36% w/w total tree nut flour. In some embodiments, the mixed tree nut powder composition comprises about 37% to about 50% w/w total tree nut flour.

In some embodiments, there is a mixed tree nut powder composition prepared by the preceding methods.

### BRIEF DESCRIPTION OF THE DRAWINGS

FIGS. 1A-1D show RP-UPLC chromatograms for walnut flour (FIG. 1A), hazelnut flour (FIG. 1B), cashew flour (FIG. 1C), and a 1:1:1 mixture of walnut flour, hazelnut flour, and cashew flour (FIG. 1D).

FIG. 2 shows an overlay of RP-UPLC chromatograms for walnut flour, hazelnut flour, and cashew flour with representative sufficiently-resolved peaks annotated for cashew (C), walnut (W), and hazelnut (H).

FIG. 3 shows a RP-UPLC chromatogram for a 1:1:1 mixture of walnut flour, hazelnut flour, and cashew flour with identified peaks annotated: (a) Cor a 14; (b) Jug r 1; (c) Ana o 3; (d) Cor a 9 (peak 1); (e) Cor a 1; (f) Jug r 4; (g) Ana o 2 (peak 1); (h) Cor a 11; (i) Ana o 2 (peak 2).

FIGS. 4A and 4B show two zoomed-in views of the same RP-UPLC chromatogram for a 1:1:1 mixture of walnut flour, hazelnut flour, and cashew flour with identified peaks annotated. The peaks for FIG. 4A correspond to (a) Cor a 14/Jug r 1; (b) Jug r 1; (c) Ana o 3; (d) Cor a 9 (peak 1); (e) Cor a 1; (f) Jug r 5; (g) Jug r 4; (h) Ana o 2 (peak 1); (i) Cor a 11; (j) Ana o 2 (peak 2). The peaks for FIG. 4B correspond to (a) Cor a 9 (peak 1); (b) Cor a 1; (c) Jug r 5; (d) Jug r 4; (e) Ana o 2 (peak 1); (f) Cor a 11; (g) Ana o 2 (peak 2).

FIGS. 5A and 5B show an SDS-PAGE analysis of walnut flour, cashew flour, and hazelnut flour before roasting (paste) and after roasting (flour) under reduced conditions (FIG. 5A) and under non-reduced conditions (FIG. 5B). Molecular weight markers (MWM) are indicated.

FIGS. 6A and 6B show a Western blot analysis of walnut flour, cashew flour, and hazelnut flour before roasting (paste) and after roasting (flour) under reduced conditions (FIG. 6A) and under non-reduced conditions (FIG. 6B). Molecular weight markers (MWM) are indicated.

FIGS. 7A and 7B shows a Western blot analysis under non-reduced conditions (FIG. 7A) and reduced conditions (FIG. 7B) of walnuts before roasting (walnut paste), after roasting at higher temperature (higher temp. flour), and at lower-temperature conditions (90° C., 110° C., and 130° C.). A molecular weight marker (MWM) and a Jug r 1 control are indicated.

FIGS. 8A and 8B shows an SDS-PAGE analysis under non-reduced conditions (FIG. 8A) and reduced conditions (FIG. 8B) of walnuts before roasting (walnut paste), after roasting at higher temperature (higher temp. flour), and at lower-temperature conditions (90° C., 110° C., and 130° C.). A molecular weight marker (MWM) and a Jug r 1 control are indicated.

### DETAILED DESCRIPTION OF THE INVENTION

In one aspect, the invention provides methods of assessing a mixed tree nut powder composition comprising tree nut flours and methods of manufacturing pharmaceutically suitable mixed tree nut powder compositions comprising tree nut flours. In another aspect, the invention provides methods of manufacturing pharmaceutical powder compositions comprising at least a first type of tree nut flour. A pharmaceutical powder composition that includes two or more tree nut flours is useful for treating individuals who are allergic to one or more species of tree nut by oral immunotherapy (OIT). By including two, three, or even more different types of tree nuts in a single powder composition, the single powder composition can be used to treat more than an allergy to a single type of nut, but can also be used for several different types of tree nut allergies. Upon mixing two or more individual tree nut flours, the proportion of each tree nut flour in the mixture, relative to other tree nut flours in the mixture, should be carefully controlled to ensure the product is safe and functions as intended. It is important to assay and verify the proportions of each tree nut flour in the mixed tree nut powder composition. As an example, if the amount of one tree nut flour is too high, the powder composition, when administered as a dose during OIT, may exceed the reactive threshold of an individual with a tree nut allergy and trigger an adverse allergic reaction. In addition, if a tree nut flour is not at a high enough amount in the powder composition, the powder composition may not be effective at triggering or maintaining a desensitization reaction to the tree nut. Simply measuring the total protein content of the mixture is not sufficient for this purpose, since the total protein content does not inform what proportion of the total protein is contributed by each tree nut flour in the mixture. The proportion of different tree nut flours in the pharmaceutical powder composition should be measured and controlled to ensure the pharmaceutical suitability of the powder composition.

Thus, described herein are quality control methods for assessing mixtures comprising two or more tree nut flours for pharmaceutical suitability. The methods include assaying the mixture of tree nut flours by liquid chromatography and assessing the mixture for pharmaceutical suitability. It has been discovered that certain chromatographic features, such as sufficiently resolved quantitative peaks, as further described herein, can be used to determine the relative proportion of a particular tree nut flour in a mixed tree nut powder composition, even in the complicated background of a mixture of two or more species of tree nut flours. By identifying pharmaceutically suitable mixed tree nut powder compositions, these compositions can be provided for use in oral immunotherapy of a tree nut allergy (or multiple tree nut allergies). The mixture of tree nut flours can be assessed as suitable for use in oral immunotherapy of tree nut allergy if the proportion of a particular tree nut flour in the mixed tree nut powder composition is within a certain range (described as a predetermined threshold, which has a lower and upper bound) relative to the other tree nut flour(s) in the mixed tree nut powder composition. The proportion is determined by measuring a ratio of the amount of a particular tree nut flour and an amount of another tree nut flour in the mixed tree nut powder composition relative to each other, and then comparing the ratio to a predetermined threshold (e.g., a range from a lower acceptance bound to an upper acceptance bound). Additional ratios can also then be measured (for example, a second ratio for an amount of a first tree nut flour and an amount of a third tree nut flour relative to each other, and a third ratio for an amount of a second tree nut flour and an amount of a third tree nut flour relative to each other). Each of these ratios, as appropriate, can be compared to their own predetermined thresholds (such as a second predetermined threshold and a third predetermined threshold). The pharmaceutical suitability of the powder composition can be assessed by comparing one or more of these ratios to corresponding predetermined thresholds.

Further described herein are methods of manufacturing a pharmaceutically suitable mixed tree nut powder composition, comprising tree nut flours, that includes a quality control method described herein. The methods involve forming the mixed tree nut powder composition, and then assaying the mixed tree nut powder composition by liquid chromatography and assessing the mixed tree nut powder composition for pharmaceutical suitability. Those mixtures assessed as pharmaceutically suitable can be packaged for use in an oral immunotherapy treatment method. Those mixtures assessed as not pharmaceutically suitable can be discarded. Certain chromatographic features, such as sufficiently resolved quantitative peaks, can be used to determine the relative proportion of a particular tree nut flour in the mixture compared to other tree nut flours in the mixture, even in the complicated background of a mixture of tree nut flours.

The invention also provides methods of manufacturing a pharmaceutical powder composition comprising at least a first type of tree nut flour. When preparing tree nut flours for use in powder compositions for the treatment of tree nut allergy, a significant challenge in processing raw tree nuts into tree nut flours suitable for pharmaceutical use in treating a tree nut allergy by OIT (as opposed to processing raw tree nuts into tree nut flours merely suitable for food purposes) is preserving the allergenic components during manufacturing. In particular, in any manufacturing steps involving heat, it is important to avoid neutralizing heat-labile allergen proteins. If a pharmaceutical powder composition does not have a protein profile similar to raw tree nuts, it may not be effective in desensitizing an individual to those raw tree nuts as part of an oral immunotherapy. Heat is often required, for example, to reduce moisture content and bioburden of tree nuts. Methods known in the art of preparing tree nut flours can alter the protein profile of the tree nut flour as compared to raw tree nuts. Methods described herein for manufacturing a pharmaceutical powder composition that include at least a first type of tree nut flour resolve this issue by roasting the one or more types of tree nut at a temperature between about 90° C. and about 140° C. (such as about 90° C. to about 130° C.) to obtain a desired moisture content. This roasting temperature is below the temperature previously used to roast tree nuts, and can help preserve the allergenicity of the powder composition. Further, the resulting pharmaceutical powder compositions have reduced moisture content, which is important to maintain the stability of the pharmaceutical powder composition. The pharmaceutical powder compositions also have protein profiles that closely resemble the protein profile of raw tree nuts, making them well-suited for use in oral immunotherapy of tree nut allergy.

### Definitions

As used herein, the singular forms “a,” “an,” and “the” include the plural references unless the context clearly dictates otherwise.

Reference to “about” a value or parameter herein includes (and describes) variations that are directed to that value or parameter per se. For example, description referring to “about X” includes description of “X”.

As used herein, a “tree nut” refers to any nut or drupe obtained from a tree. Tree nuts include, but are not limited to, walnuts (Juglans spp.), almonds (Prunus dulcis), pistachios (Pistacia vera L.), cashews (Anacardium occidentale), pecans (Carya illinoensis), hazelnut (Corylus spp.), macadamias (Macadamia spp.), Brazil nuts (Bertholletia excelsa), pine nuts (Pinus spp.), beechnuts (Fagus spp.), butternuts (Juglans cinerea), chestnuts (Castanea spp.), chinquapins (Castanea pumila), coconuts (Cocos nucifera L.), ginkgo nuts (Ginkgo biloba L.), hickory nuts (Carya spp.), lichee nuts (Litchi chinensis Sonn.), pili nuts (Canarium ovatum), and sheanuts (Vitellaria paradoxa C. F. Gaertn), and any botanical varieties, forms, or cultivars thereof.

As used herein, a “flour” or a “tree nut flour” refers to a powder made by grinding a tree nut. Generally, but not necessarily, a given tree nut can be defatted.

As used herein, “defatted tree nut flour” and “partially defatted tree nut flour” are used synonymously to mean that a tree nut flour has reduced fat content (such as reduced oil content) relative to the corresponding raw tree nut.

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The disclosures of all publications, patents, and patent applications referred to herein are each hereby incorporated by reference in their entireties. To the extent that any reference incorporated by reference conflicts with the instant disclosure, the instant disclosure shall control.

### Quality Control of Mixed Tree Nut Powder Compositions

Powder compositions comprising two or more tree nut flours are useful for treating allergies to tree nuts by oral immunotherapy (OIT). However, upon mixing two or more individual tree nut flours, it is beneficial to ensure the relative proportion of each tree nut flour in the mixture is within an acceptance range. Measuring the total protein content of the powder mixture comprising two or more individual tree nut flours is generally insufficient for quality control because one tree nut flour may be at an unexpectedly higher or lower proportion relative to another tree nut flour in the composition, even though the total protein content may be the same as if they were in the correct proportions. If one tree nut flour is present at too high of an amount, the powder composition may trigger a dangerous allergic reaction in an individual having an allergy to that tree nut flour. If the tree nut flour is instead present at too low of an amount, the powder composition may not be effective in triggering or maintaining desensitization to the tree nut flour. For example, if a mixture comprises walnut flour and cashew flour for use in treating walnut allergy, if the walnut flour is above the acceptable level then the mixture may trigger an allergic reaction when administered to an individual having a walnut allergy. On the other hand, if the walnut flour is below the acceptable level, then the mixture may not be effective at inducing or maintaining desensitization to walnut allergy when used as part of an oral immunotherapy to walnut allergy. Suitable mixtures may be formulated, packaged, and/or used to treat a subject with a tree nut allergy by oral immunotherapy, wherein unsuitable mixtures may be discarded.

A quality control process has been developed which allows the relative proportion(s) of one or more tree nut flours in a mixed tree nut powder composition comprising two or more tree nut flours to be determined by liquid chromatography. Once the proportion(s) of the one or more tree nut flours in the mixed tree nut powder composition are assessed, they can be compared to an acceptance threshold (i.e., a predetermined threshold) and the mixed tree nut powder composition can be assessed as pharmaceutically suitable or not pharmaceutically suitable based on the measured proportion(s) as compared to the predetermined threshold(s).

In some embodiments, there is a method of assessing a mixed tree nut powder composition for pharmaceutical suitability, wherein the mixed tree nut powder composition comprises at least a first tree nut flour and a second tree nut flour, comprising assaying the mixed tree nut powder composition by liquid chromatography to determine a ratio for an amount of the first tree nut flour and an amount of the second tree nut flour relative to each other; assessing whether the mixed tree nut powder composition is pharmaceutically suitable, wherein the mixed tree nut powder composition is pharmaceutically suitable only if the ratio is within a predetermined threshold. In some embodiments, the method further comprises packaging the mixed tree nut powder composition if the mixed tree nut powder composition is assessed as pharmaceutically suitable. In some embodiments, the mixed tree nut powder composition is packaged in a pharmaceutically acceptable dosage form, such as a capsule, a tablet, a sachet, a pouch, or a sprinkle capsule. In some embodiments, the tree nut flour is assessed as pharmaceutically unsuitable, wherein the mixed tree nut powder composition is pharmaceutically unsuitable if the ratio is not within a predetermined threshold. In some embodiments, if the mixed tree nut powder composition is discarded if assessed as pharmaceutically unsuitable. In some embodiments, the mixed tree nut powder composition further comprises one or more pharmaceutically acceptable excipients. In some embodiments, the liquid chromatography is ultra-high-performance liquid chromatography (UPLC). In some embodiments, the UPLC is reversed-phase UPLC (RP-UPLC). In some embodiments, the predetermined threshold is a range set between about 0.75 and about 1.25. In some embodiments, the first tree nut flour is cashew flour. In some embodiments, the first tree nut flour is hazelnut flour. In some embodiments, the first tree nut flour is walnut flour. In some embodiments, the second tree nut flour is cashew flour. In some embodiments, the second tree nut flour is hazelnut flour. In some embodiments, the second tree nut flour is walnut flour.

In some embodiments, there is a method of assessing a mixed tree nut powder composition for pharmaceutical suitability, wherein the mixed tree nut powder composition comprises at least a first tree nut flour, a second tree nut flour, and a third tree nut flour, comprising assaying the mixed tree nut powder composition by liquid chromatography to determine a ratio for an amount of the first tree nut flour and an amount of the second tree nut flour relative to each other; determining a second ratio for the amount of the first tree nut flour and the amount of the third tree nut flour relative to each other; determining a third ratio for the amount of the second tree nut flour and the amount of the third tree nut flour relative to each other; and assessing whether the mixed tree nut flour is pharmaceutically suitable, wherein the mixed tree nut powder composition is pharmaceutically suitable only if the ratio is within a predetermined threshold, the second ratio is within a second predetermined threshold, and the third ratio is within a third predetermined threshold. In some embodiments, the method further comprises packaging the mixed tree nut powder composition if the mixed tree nut powder composition is assessed as pharmaceutically suitable. In some embodiments, the mixed tree nut powder composition is packaged in a pharmaceutically acceptable dosage form. In some embodiments, the mixed tree nut powder composition is assessed as pharmaceutically unsuitable, wherein the mixed tree nut powder composition is pharmaceutically unsuitable if at least one of the ratios (such as at least one of the ratio, the second ratio, or the third ratio) is/are not within a predetermined threshold. In some embodiments, the mixed tree nut powder composition is discarded if assessed as pharmaceutically unsuitable. In some embodiments, the mixed tree nut powder composition further comprises one or more pharmaceutically acceptable excipients. In some embodiments, the liquid chromatography is ultra-high-performance liquid chromatography (UPLC). In some embodiments, the UPLC is reversed-phase UPLC (RP-UPLC). In some embodiments, the predetermined threshold is a range set between about 0.75 and about 1.25. In some embodiments, the second predetermined threshold is a range set between about 0.75 and about 1.25. In some embodiments, the third predetermined threshold is a range set between about 0.75 and about 1.25. In some embodiments, the first tree nut flour is cashew flour. In some embodiments, the first tree nut flour is hazelnut flour. In some embodiments, the first tree nut flour is walnut flour. In some embodiments, the second tree nut flour is cashew flour. In some embodiments, the second tree nut flour is hazelnut flour. In some embodiments, the second tree nut flour is walnut flour. In some embodiments, the third tree nut flour is cashew flour. In some embodiments, the third tree nut flour is hazelnut flour. In some embodiments, the third tree nut flour is walnut flour. In some embodiments, the first tree nut flour is walnut flour, the second tree nut flour is hazelnut flour, and the third tree nut flour is cashew flour.

In some embodiments, there is a method of manufacturing a pharmaceutically suitable mixed tree nut powder composition, comprising forming the mixed tree nut powder composition, comprising mixing at least a first tree nut flour and a second tree nut flour; assaying the mixed tree nut powder composition by liquid chromatography to determine a ratio for an amount of the first tree nut flour and an amount of the second tree nut flour relative to each other; assessing whether the mixed tree nut powder composition is pharmaceutically suitable, wherein the mixed tree nut powder composition is pharmaceutically suitable only if the ratio is within a predetermined threshold; and packaging the mixed tree nut powder composition if assessed to be pharmaceutically suitable. In some embodiments, the mixed tree nut powder composition is packaged in a pharmaceutically acceptable dosage form. In some embodiments, the liquid chromatography is ultra-high-performance liquid chromatography (UPLC). In some embodiments, the UPLC is reversed-phase UPLC (RP-UPLC). In some embodiments, the predetermined threshold is a range set between about 0.75 and about 1.25. In some embodiments, forming the mixed tree nut powder composition comprises mixing with one or more pharmaceutically acceptable excipients. In some embodiments, forming the mixed tree nut powder composition comprises simultaneously mixing the first tree nut flour and the second tree nut flour with one or more pharmaceutically acceptable excipients. In some embodiments, forming the mixed tree nut powder composition comprises mixing the first tree nut flour and the second tree nut flour to form a tree nut flour mixture, and then mixing the tree nut flour mixture with one or more pharmaceutically acceptable excipients. In some embodiments, the mixed tree nut powder composition is packaged in a pharmaceutically acceptable dosage form, such as a capsule, a tablet, a sachet, a pouch, or a sprinkle capsule. In some embodiments, the first tree nut flour is cashew flour. In some embodiments, the first tree nut flour is hazelnut flour. In some embodiments, the first tree nut flour is walnut flour. In some embodiments, the second tree nut flour is cashew flour. In some embodiments, the second tree nut flour is hazelnut flour. In some embodiments, the second tree nut flour is walnut flour.

In some embodiments, there is a method of manufacturing a pharmaceutically suitable mixed tree nut powder composition, comprising forming the mixed tree nut powder composition, comprising mixing at least a first tree nut flour, a second tree nut flour, and a third tree nut flour; assaying the mixed tree nut powder composition by liquid chromatography to determine a ratio for an amount of the first tree nut flour and an amount of the second tree nut flour relative to each other, determining a second ratio for the amount of the first tree nut flour and an amount for the third tree nut flour relative to each other, determining a third ratio for the amount of the second tree nut flour and an amount for the third tree nut flour relative to each other; assessing whether the mixed tree nut powder composition is pharmaceutically suitable, wherein the mixed tree nut powder composition is pharmaceutically suitable only if the ratio is within a predetermined threshold, the second ratio is within a second predetermined threshold, and the third ratio is within a third predetermined threshold; and packaging the mixed tree nut powder composition if assessed to be pharmaceutically suitable. In some embodiments, the mixed tree nut powder composition is packaged in a pharmaceutically acceptable dosage form, such as a capsule, a tablet, a sachet, a pouch, or a sprinkle capsule. In some embodiments, the liquid chromatography is ultra-high-performance liquid chromatography (UPLC). In some embodiments, the UPLC is reversed-phase UPLC (RP-UPLC). In some embodiments, the predetermined threshold is a range set between about 0.75 and about 1.25. In some embodiments, the second predetermined threshold is a range set between about 0.75 and about 1.25. In some embodiments, the third predetermined threshold is a range set between about 0.75 and about 1.25. In some embodiments, forming the mixed tree nut powder composition comprises mixing with one or more pharmaceutically acceptable excipients. In some embodiments, forming the mixed tree nut powder composition comprises simultaneously mixing the first tree nut flour, the second tree nut flour, and the third tree nut flour with one or more pharmaceutically acceptable excipients. In some embodiments, forming the mixed tree nut powder composition comprises mixing the first tree nut flour, the second tree nut flour, and the third tree nut flour to form a tree nut flour mixture, and then mixing the tree nut flour mixture with one or more pharmaceutically acceptable excipients. In some embodiments, assaying comprises measuring a plurality of peak areas of sufficiently resolved quantitative peaks. In some embodiments, the first tree nut flour is cashew flour. In some embodiments, the first tree nut flour is hazelnut flour. In some embodiments, the first tree nut flour is walnut flour. In some embodiments, the second tree nut flour is cashew flour. In some embodiments, the second tree nut flour is hazelnut flour. In some embodiments, the second tree nut flour is walnut flour. In some embodiments, the third tree nut flour is cashew flour. In some embodiments, the third tree nut flour is hazelnut flour. In some embodiments, the third tree nut flour is walnut flour. In some embodiments, the first tree nut flour is walnut flour, the second tree nut flour is hazelnut flour, and the third tree nut flour is cashew flour.

### Liquid Chromatography

The quality control method includes assaying mixed tree nut powder compositions comprising tree nut flours by liquid chromatography to determine a ratio for an amount of a tree nut flour in a mixed tree nut powder composition relative to an amount of one or more other tree nut flours in the mixed tree nut powder composition. Liquid chromatography is a technique that separates samples into components based on the interactions of a sample with mobile and stationary phases. In the case where a mixed tree nut powder composition comprises exactly two species of tree nut flours, then a single ratio may be obtained (a ratio for an amount of the first tree nut flour and an amount of a second tree nut flour relative to each other). The ratio may then be compared to a predetermined threshold to assess the mixed tree nut powder composition as pharmaceutically suitable only if the ratio is within the predetermined threshold. If the ratio is not within the predetermined threshold, the composition is assessed as not pharmaceutically suitable. In some embodiments, the mixed tree nut powder composition comprises at least a first tree nut flour and a second tree nut flour. In some embodiments, the mixed tree nut powder composition comprises a tree nut flour portion consisting of a first tree nut flour and a second tree nut flour.

In the case where more than two species of tree nut flours are in the mixed tree nut powder composition, then a ratio can be obtained for each individual tree nut flour relative to each other tree nut flour in the mixed tree nut powder composition. For example, in a mixture of exactly three types of tree nut flours, a ratio for the amount of the first tree nut flour and the amount of the second tree nut flour relative to each other, a second ratio for the amount of the first tree nut flour and the amount of the third tree nut flour relative to each other, and a third ratio for the amount of the second tree nut flour and the amount of the third tree nut flour relative to each other may be determined. Each of the ratio, the second ratio, and the third ratio can then be compared to predetermined thresholds (such as a predetermined threshold, a second predetermined threshold, and a third predetermined threshold) to assess the mixed tree nut powder composition as pharmaceutically suitable only if ratios are each within their respective predetermined thresholds. If at least one of the ratio, the second ratio, or the third ratio are not within their respective predetermined thresholds, the mixed tree nut powder composition is assessed as not pharmaceutically suitable. In some embodiments, the mixed tree nut powder composition comprises at least a first tree nut flour, a second tree nut flour, and a third tree nut flour. In some embodiments, the mixed tree nut powder composition comprises a tree nut flour portion consisting of a first tree nut flour, a second tree nut flour, and a third tree nut flour.

The liquid chromatography assay involves measuring one or more peak areas of sufficiently resolved quantitative peaks. A “sufficiently resolved quantitative peak” should be substantially separated such that the peak area is substantially correlated with the amount of a particular tree nut flour in the mixed tree nut powder composition.

For example, in a mixed tree nut powder composition comprising three types of tree nut flour, a sufficiently resolved quantitative peak corresponding to a first tree nut flour (out of the three tree nut flours in the composition) is one whose peak area substantially correlates with the amount of the first tree nut flour in the composition. For further illustration, if there is a mixed tree nut powder composition comprising three distinct types of tree nut flours each at a proportion of 1:1:1, and a peak area assigned to the first tree nut flour has a peak area of 1.0, then if the first tree nut flour is halved (to give a proportion of 0.5:1:1, instead of 1:1:1), then the peak area should also be reduced to about 0.5. Conversely, if the first tree nut flour is doubled (to give a proportion of 2:1:1, instead of 1:1:1), then the peak area should also be increased to about 2.0. It is not expected that this correlation is exact, and instead a sufficiently resolved quantitative peak should be substantially correlated with the amount of a particular tree nut flour. In other words, the meaning of “quantitative” in the term “sufficiently resolved quantitative peak” means that the peak can be used to determine the amount of an associated tree nut flour in a mixed tree nut powder composition, relative to another tree nut flour in the composition. In some embodiments, a peak is a sufficiently resolved quantitative peak if it has a correlation of at least a factor of 0.8 to 1.2 of a predicted value. In the above example of 0.5:1:1, the predicted peak area is 0.5 (i.e., the ratio of the amount of the first tree nut flour and the amount of the second tree nut flour relative to each other), and a peak is a sufficiently resolved quantitative peak if the peak area corresponding to that ratio for the 0.5:1:1 mixture is between 0.4 and 0.6. In the above example of 2:1:1, the predicted peak area is 2.0 (i.e., the ratio of the amount of the first tree nut flour and the amount of the second tree nut flour relative to each other), and a peak is a sufficiently resolved quantitative peak if the peak area corresponding to that ratio for the 2:1:1 mixture is between 1.6 and 2.4. In some embodiments, a peak is a sufficiently resolved quantitative peak if it has a correlation of at least a factor of 0.8 to 1.2 of a predicted value, such as 0.8 to 1.2, 0.85 to 1.15, 0.90 to 1.10, or 0.95 to 1.05 of a predicted value.

A sufficiently resolved quantitative peak need not be perfectly resolved from neighboring peaks to be useful for the methods described herein. Instead, the methods described herein rely on measured peaks being “sufficiently resolved” from neighboring peaks. The resolution of a sufficiently resolved quantitative peak can be assessed by the resolution, “R”, of the peak. The R of a peak is a quantitative measure of how well two peaks can be differentiated in the chromatographic separation and depends on the retention time of the two peaks divided by the combined widths of the elution peaks. In some embodiments, the resolution is determined by the equation R=(2(t2−t1)/(W2+W1)), where t2 and t1 are the retention times of two peaks, and W2 and W1 are the corresponding widths at the bases of the peaks obtained by extrapolating the relatively straight sides of the peaks to the baseline. In some embodiments, the resolution is determined by a method described in the U.S. Pharmacopeia, such as USP29 (The United States Pharmacopeia, USP29-NF24 2006). In some embodiments, a peak is a sufficiently resolved quantitative peak if the resolution of the peak is at least 0.6 as compared to any neighboring peak, such as at least 0.6, 0.65, 0.70, 0.75, 0.80, 0.85, 0.90, 0.95, or 1.0 as compared to any neighboring peak.

Different species of tree nut flours have unique chromatographic profiles. The liquid chromatography conditions may need to be adjusted depending on the identity and quantity of tree nut flours in the mixed tree nut powder composition, and a peak that is a sufficiently resolved quantitative peak in one condition may not be a sufficiently resolved quantitative peak, as used herein, under alternative conditions. Thus, in some embodiments of any of the described methods, the method further comprises identifying one or more sufficiently resolved quantitative peaks. One or more sufficiently resolved quantitative peaks may be identified for any of the tree nut flours in the mixed tree nut powder composition. In some embodiments of any of the described methods, the method further comprises identifying one or more sufficiently resolved quantitative peaks for a first tree nut flour. In some embodiments of any of the described methods, the method further comprises identifying one or more sufficiently resolved quantitative peaks for a second tree nut flour. In some embodiments of any of the described methods, the method further comprises identifying one or more sufficiently resolved quantitative peaks for a third tree nut flour. In some embodiments of any of the described methods, the method further comprises identifying one or more sufficiently resolved quantitative peaks for a first tree nut flour and a second tree nut flour. In some embodiments of any of the described methods, the method further comprises identifying one or more sufficiently resolved quantitative peaks for each of a first tree nut flour, a second tree nut flour, and a third tree nut flour. In some embodiments of any of the described methods, the method further comprises identifying one or more sufficiently resolved quantitative peaks for at least one of a first tree nut flour, a second tree nut flour, and a third tree nut flour.

More than one sufficiently resolved quantitative peak may be assessed for a particular tree nut flour in the mixed tree nut powder composition. For example, in a mixed tree nut powder composition comprising at least a first tree nut flour and a second tree nut flour, at least one sufficiently resolved quantitative peak may be measured for the first tree nut flour and at least one sufficiently resolved quantitative peak may be measured for the second tree nut flour. In a mixed tree nut powder composition comprising at least a first tree nut flour, a second tree nut flour, and a third tree nut flour, at least one sufficiently resolved quantitative peak may be measured for the first tree nut flour, at least one sufficiently resolved quantitative peak may be measured for the second tree nut flour, and at least one sufficiently resolved quantitative peak may be measured the third tree nut flour. In some embodiments, at least two sufficiently resolved quantitative peaks are measured for the first tree nut flour. In some embodiments, at least two sufficiently resolved quantitative peaks are measured for the second tree nut flour. In some embodiments, at least two sufficiently resolved quantitative peaks are measured for the third tree nut flour. In some embodiments, at least one sufficiently resolved quantitative peak is measured for each of the first tree nut flour and the second tree nut flour. In some embodiments, at least one sufficiently resolved quantitative peak is measured for each of the first tree nut flour and the second tree nut flour, and at least two sufficiently resolved quantitative peaks are measured for the first tree nut flour or the second tree nut flour. In some embodiments, at least one sufficiently resolved quantitative peak is measured for each of the first tree nut flour, the second tree nut flour, and the third tree nut flour, and at least two sufficiently resolved quantitative peaks are measured for the first tree nut flour, the second tree nut flour, or the third tree nut flour.

It is further understood that a sufficiently resolved quantitative peak may be substantially, but not entirely, associated with a particular protein in a tree nut flour. In other words, it is possible that two or more distinct proteins in a particular type of tree nut flour may have identical or highly similar retention times. As used herein, a sufficiently resolved quantitative peak comprising a peak “corresponding to” a particular protein means that the particular protein comprises a substantial portion of the protein eluting from the peak area. In some embodiments, a sufficiently resolved quantitative peak comprises a peak corresponding to a particular protein if the particular protein is greater than about 50% of the total protein eluting from a peak area, such as at least about any of 55%, 60%, 65%, 70%, 75%, 80%, 85%, 90%, 95%, or 99% of the protein eluting from the peak area. Additionally, a sufficiently resolved quantitative peak comprising a peak corresponding to a particular protein may be associated with an isomer or conformer of the protein. As used herein, a sufficiently resolved quantitative peak comprising a peak corresponding to a particular protein includes a single peak corresponding to one or more conformers or isomers of that particular protein. For illustration, in an experiment using walnut flour, Jug r 1 was found to elute at retention time of about 1.788 minutes (RT=1.788) and RT=1.88 under certain RP-UPLC conditions. Either of the RT=1.788 or RT=1.88, for the purposes of this application, may be a sufficiently resolved quantitative peak comprising a peak corresponding to Jug r 1 if the peak otherwise meets the requirements of a sufficiently resolved quantitative peak described herein. Similarly, a sufficiently resolved quantitative peak may comprise a peak corresponding to a particular tree nut flour, which means that a substantial portion of the protein(s) eluting from the peak area are from the particular tree nut flour. In some embodiments, a sufficiently resolved quantitative peak comprises a peak corresponding to a particular tree nut flour if greater than 50% of the protein eluting from the peak is from the said particular tree nut flour, such as at least 55%, 60%, 65%, 70%, 75%, 80%, 85%, 90%, 95%, or 99% of the protein eluting from the peak area is from the said particular tree nut flour.

The sufficiently resolved quantitative peaks may comprise a peak corresponding to any protein in a tree nut flour, including, for example, a tree nut allergen protein. In some embodiments, the one or more tree nut allergen proteins comprise a metabolic protein. In some embodiments, the one or more tree nut allergen proteins comprise a seed storage protein. In some embodiments, the seed storage protein is a member of the prolamin superfamily (such as the 2S albumins) or the cupin superfamily (such as 11S globulins and 7S globulins). In some embodiments, the one or more tree nut allergen proteins comprise a lipid transfer protein. In some embodiments, the one or more tree nut allergen proteins comprise a profilin. In some embodiments, the one or more tree nut allergen proteins comprise a pathogenesis-related protein. In some embodiments, the one or more tree nut allergen proteins comprise a hevein. In some embodiments, the one or more tree nut allergen proteins are selected from the listing consisting of Cor a 1, Cor a 2, Cor a 8, Cor a 9, Cor a 11, Cor a 12, Cor a 13, Cor a 14, Ana o 1, Ana o 2, Ana o 3, Pis v 1, Pis v 2, Pis v 3, Pis v 5, Jug r 1, Jug r 2, Jug r 4, Jug r 6, Jug r 3, Jug r 5, Car i 1, Car i 2, Car i 4, Pru du 6, Pru du 3, Pru du 4, Pin p 1, Ber e 1, and Ber e 2.

In some embodiments, the one or more sufficiently resolved quantitative peaks comprise one or more peaks corresponding to one or more tree nut proteins selected from the group consisting of a 11S globulin, a 7S globulin, a lipid transfer protein, a Bet v 1-like protein, a profilin protein, and an oleosin protein. In some embodiments, the one or more sufficiently resolved quantitative peaks comprise one or more peaks corresponding to one or more cashew proteins selected from the group consisting of a 11S globulin, a 7S globulin, a lipid transfer protein, a Bet v 1-like protein, a profilin protein, and an oleosin protein. In some embodiments, the one or more sufficiently resolved quantitative peaks comprise one or more peaks corresponding to one or more hazelnut proteins selected from the group consisting of a 11S globulin, a 7S globulin, a lipid transfer protein, a Bet v 1-like protein, a profilin protein, and an oleosin protein. In some embodiments, the one or more sufficiently resolved quantitative peaks comprise one or more peaks corresponding to one or more walnut proteins selected from the group consisting of a 11S globulin, a 7S globulin, a lipid transfer protein, a Bet v 1-like protein, a profilin protein, and an oleosin protein.

In some embodiments, the one or more sufficiently resolved quantitative peaks comprise a peak corresponding to a tree nut flour selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour. In some embodiments, the one or more sufficiently resolved quantitative peaks comprise a peak corresponding to walnut flour. In some embodiments, the one or more sufficiently resolved quantitative peaks comprise a peak corresponding to hazelnut flour. In some embodiments, the one or more sufficiently resolved quantitative peaks comprise a peak corresponding to cashew flour. In some embodiments, the one or more sufficiently resolved quantitative peaks comprise a peak corresponding to hazelnut flour, a peak corresponding to cashew flour, and a peak corresponding to walnut flour.

In some embodiments, the one or more sufficiently resolved quantitative peaks comprise a peak corresponding to a 2S albumin of walnut. In some embodiments, the one or more sufficiently resolved quantitative peaks comprise a peak corresponding to a 2S albumin of hazelnut. In some embodiments, the one or more sufficiently resolved quantitative peaks comprise a peak corresponding to a 2S albumin of cashew. In some embodiments, the one or more sufficiently resolved quantitative peaks comprise a peak corresponding to Jug r 1. In some embodiments, the one or more sufficiently resolved quantitative peaks comprise a peak corresponding to Jug r 2. In some embodiments, the one or more sufficiently resolved quantitative peaks comprise a peak corresponding to Cor a 9. In some embodiments, the one or more sufficiently resolved quantitative peaks comprise a peak corresponding to Cor a 14. In some embodiments, the one or more sufficiently resolved quantitative peaks comprise a peak corresponding to Ana o 3. In some embodiments, the one or more sufficiently resolved quantitative peaks comprise a peak corresponding to Ana o 2.

### Predetermined Thresholds

The quality control methods described herein can include comparing ratios of amounts of tree nut flours relative to each other to predetermined thresholds to assess the mixed tree nut powder compositions as pharmaceutically suitable or not pharmaceutically suitable. The predetermined thresholds are expressed as a range, comprising a lower bound and an upper bound. The lower bound is intended to ensure that there is a sufficient amount of a particular tree nut flour to trigger a desensitization reaction during oral immunotherapy. The upper bound is intended to ensure that there is not so much of a particular tree nut flour as to exceed the reactive threshold of an individual upon administration of a dose of the mixed tree nut flour during oral immunotherapy such that an adverse allergic reaction is likely to be triggered. Between the lower and upper bound is a target tree nut flour ratio of two tree nut flours in the mixture. The predetermined threshold for a ratio of an amount of one tree nut flour to an amount of another tree nut flour relative to each other depends on the target ratio. For example, in a mixture comprising three species of tree nut flours, if the target proportion is 1:1:1, then the target ratio of an amount of the first tree nut flour and an amount of the second tree nut flour relative to each other is 1.0. However, if the target proportion is 0.5:1:1, then the target ratio of an amount of the first tree nut flour and an amount of the second tree nut flour relative to each other is 0.5. In this example of a tree nut flour mixture comprising three distinct tree nut flours, there are three possible ratios which may be determined (i.e., a ratio, a second ratio, and a third ratio), which can each be compared to their own predetermined thresholds (i.e., a predetermined threshold, a second predetermined threshold, and a third predetermined threshold). In the event one or more the ratios do not fall within their respective predetermined thresholds, the mixed tree nut powder composition is assessed as not pharmaceutically suitable and may be discarded. The mixed tree nut powder composition is assessed as pharmaceutically suitable only if one or more of the ratios is/are within the predetermined thresholds, whereupon the mixed tree nut powder composition, in some embodiments, may be packaged for use in oral immunotherapy.

In some embodiments, the predetermined threshold (such as the predetermined threshold, the second predetermined threshold, the third predetermined threshold, or any other predetermined threshold) is a range set between about 0.25 and about 3.00, such as about 0.25 to about 0.50, about 0.50 to about 0.75, about 0.75 to about 1.00, about 1.00 to about 1.25, about 1.25 to about 1.50, about 1.50 to about 1.75, about 1.75 to about 2.0, about 2.0 to about 2.25, about 2.25 to about 2.50, about 2.50 to about 2.75, about 2.75 to about 3.0, about 0.25 to about 0.75, about 0.50 to about 1.0, about 0.75 to about 1.25, about 1.0 to about 1.50, about 1.25 to about 1.5, about 1.5 to about 2.0, about 1.75 to about 2.25, about 2.0 to about 2.50, about 2.25 to about 2.75, or about 2.5 to about 3.0, any ranges therebetween. In some embodiments, the predetermined threshold is a range set between about 0.25 and 0.50. In some embodiments, the predetermined threshold is a range set between about 0.30 and about 0.60. In some embodiments, the predetermined threshold is a range set between about 0.50 and about 0.75. In some embodiments, the predetermined threshold is a range set between about 0.75 and about 1.00. In some embodiments, the predetermined threshold is a range set between about 0.80 and about 1.20. In some embodiments, the predetermined threshold is a range set between about 0.90 and about 1.10. In some embodiments, the predetermined threshold is a range set between about 1.00 and about 1.25. In some embodiments, the predetermined threshold is a range set between about 0.75 and about 1.25. In some embodiments, the predetermined threshold is a range set between about 0.80 and about 1.20. In some embodiments, the predetermined threshold is a range set between about 0.85 and about 1.15. In some embodiments, the predetermined threshold is a range set between about 0.90 and about 1.10. In some embodiments, the predetermined threshold is a range set between about 0.95 and about 1.05. In any of these described embodiments, the predetermined threshold can refer to a predetermined threshold (meaning a first predetermined threshold or the only predetermined threshold, as evident by the context it is used), a second predetermined threshold, a third predetermined threshold, or any other predetermined threshold.

In some embodiments, the method comprises assaying a mixed tree nut powder composition by liquid chromatography, wherein the mixed tree nut powder composition comprises at least a first tree nut flour and a second tree nut flour, wherein the method comprises determining a ratio for an amount of the first tree nut flour and an amount of the second tree nut flour relative to each other; and assessing whether the mixed tree nut powder composition is pharmaceutically suitable, wherein the mixed tree nut powder composition is pharmaceutically suitable only if the ratio is within a predetermined threshold, wherein the predetermined threshold is a range set between about 0.75 and about 1.25.

In some embodiments, the method comprises assaying a mixed tree nut powder composition by liquid chromatography, wherein the mixed tree nut powder composition comprises at least a first tree nut flour and a second tree nut flour, wherein the method comprises determining a ratio for an amount of the first tree nut flour and an amount of the second tree nut flour relative to each other; and assessing whether the mixed tree nut powder composition is pharmaceutically suitable, wherein the mixed tree nut powder composition is pharmaceutically suitable only if the ratio is within a predetermined threshold, wherein the predetermined threshold is a range set between about 0.80 and about 1.20.

In some embodiments, the method comprises assaying a mixed tree nut powder composition by liquid chromatography, wherein the mixed tree nut powder composition comprises at least a first tree nut flour and a second tree nut flour, wherein the method comprises determining a ratio for an amount of the first tree nut flour and an amount of the second tree nut flour relative to each other; and assessing whether the mixed tree nut powder composition is pharmaceutically suitable, wherein the mixed tree nut powder composition is pharmaceutically suitable only if the ratio is within a predetermined threshold, wherein the predetermined threshold is a range set between about 0.90 and about 1.10.

In some embodiments, the method comprises assaying a mixed tree nut powder composition by liquid chromatography, wherein the mixed tree nut powder composition comprises at least a first tree nut flour, a second tree nut flour, and a third tree nut flour, wherein the method comprises determining a ratio for an amount of the first tree nut flour and an amount of the second tree nut flour relative to each other, determining a second ratio for the amount of the first tree nut flour and the third tree nut flour relative to each other, and determining a third ratio for the amount of the second tree nut flour and the third tree nut flour relative to each other; and assessing whether the mixed tree nut powder composition is pharmaceutically suitable, wherein the mixed tree nut powder composition is pharmaceutically suitable only if the ratio is within a predetermined threshold, the second ratio is within a second predetermined threshold, and the third ratio is within a third predetermined threshold; wherein the predetermined threshold is a range set between about 0.75 and about 1.25, the second predetermined threshold is a range set between about 0.75 and about 1.25, and the third predetermined threshold is a range set between about 0.75 and about 1.25.

In some embodiments, the method comprises assaying a mixed tree nut powder composition by liquid chromatography, wherein the mixed tree nut powder composition comprises at least a first tree nut flour, a second tree nut flour, and a third tree nut flour, wherein the method comprises determining a ratio for an amount of the first tree nut flour and an amount of the second tree nut flour relative to each other, determining a second ratio for the amount of the first tree nut flour and an amount of the third tree nut flour relative to each other, and determining a third ratio for the amount of the second tree nut flour and the amount of the third tree nut flour relative to each other; and assessing whether the mixed tree nut powder composition is pharmaceutically suitable, wherein the mixed tree nut powder composition is pharmaceutically suitable only if the ratio is within a predetermined threshold, the second ratio is within a second predetermined threshold, and the third ratio is within a third predetermined threshold; wherein the predetermined threshold is a range set between about 0.80 and about 1.20, the second predetermined threshold is a range set between about 0.80 and about 1.20, and the third predetermined threshold is a range set between about 0.80 and about 1.20.

In some embodiments, the method comprises assaying a mixed tree nut powder composition by liquid chromatography, wherein the mixed tree nut powder composition comprises at least a first tree nut flour, a second tree nut flour, and a third tree nut flour, wherein the method comprises determining a ratio for an amount of the first tree nut flour and an amount of the second tree nut flour relative to each other, determining a second ratio for the amount of the first tree nut flour and the amount of the third tree nut flour relative to each other, and determining a third ratio for the amount of the second tree nut flour and the amount of the third tree nut flour relative to each other; and assessing whether the mixed tree nut powder composition is pharmaceutically suitable, wherein the mixed tree nut powder composition is pharmaceutically suitable only if the ratio is within a predetermined threshold, the second ratio is within a second predetermined threshold, and the third ratio is within a third predetermined threshold; wherein the predetermined threshold is a range set between about 0.90 and about 1.10, the second predetermined threshold is a range set between about 0.90 and about 1.10, and the third predetermined threshold is a range set between about 0.90 and about 1.10.

In some embodiments, the method comprises assaying a mixed tree nut powder composition by liquid chromatography, wherein the mixed tree nut powder composition comprises walnut flour, hazelnut flour, and cashew flour, wherein the method comprises determining a ratio for an amount of walnut flour and an amount of hazelnut flour relative to each other, determining a second ratio for the amount of walnut flour and the amount of cashew flour relative to each other, and determining a third ratio for the amount of hazelnut flour and the amount of cashew flour relative to each other; and assessing whether the mixed tree nut powder composition is pharmaceutically suitable, wherein the mixed tree nut powder composition is pharmaceutically suitable only if the ratio is within a predetermined threshold, the second ratio is within a second predetermined threshold, and the third ratio is within a third predetermined threshold; wherein the predetermined threshold is a range set between about 0.75 and about 1.25, the second predetermined threshold is a range set between about 0.75 and about 1.25, and the third predetermined threshold is a range set between about 0.75 and about 1.25.

In some embodiments, the method comprises assaying a mixed tree nut powder composition by liquid chromatography, wherein the mixed tree nut powder composition comprises walnut flour, hazelnut flour, and cashew flour, wherein the method comprises determining a ratio for an amount of walnut flour and an amount of hazelnut flour relative to each other, determining a second ratio for the amount of walnut flour and the amount of cashew flour relative to each other, and determining a third ratio for the amount of hazelnut flour and the amount of cashew flour relative to each other; and assessing whether the mixed tree nut powder composition is pharmaceutically suitable, wherein the mixed tree nut powder composition is pharmaceutically suitable only if the ratio is within a predetermined threshold, the second ratio is within a second predetermined threshold, and the third ratio is within a third predetermined threshold; wherein the predetermined threshold is a range set between about 0.80 and about 1.20, the second predetermined threshold is a range set between about 0.80 and about 1.20, and the third predetermined threshold is a range set between about 0.80 and about 1.20.

In some embodiments, the method comprises assaying a mixed tree nut powder composition by liquid chromatography, wherein the mixed tree nut powder composition comprises walnut flour, hazelnut flour, and cashew flour, wherein the method comprises determining a ratio for the amount of walnut flour and the amount of hazelnut flour relative to each other, determining a second ratio for the amount of walnut flour and the amount of cashew flour relative to each other, and determining a third ratio for the amount of hazelnut flour and the amount of cashew flour relative to each other; and assessing whether the mixed tree nut powder composition is pharmaceutically suitable, wherein the mixed tree nut powder composition is pharmaceutically suitable only if the ratio is within a predetermined threshold, the second ratio is within a second predetermined threshold, and the third ratio is within a third predetermined threshold; wherein the predetermined threshold is a range set between about 0.90 and about 1.10, the second predetermined threshold is a range set between about 0.90 and about 1.10, and the third predetermined threshold is a range set between about 0.90 and about 1.10.

### Tree Nut Flours

The quality control methods described herein pertain to methods of assessing and methods of manufacturing mixed tree nut powder compositions. The mixed tree nut powder compositions comprise two or more distinct tree nut flours, such as at least two tree nut flours, at least three nut flours, or at least four tree nut flours. In any of the described embodiments, when a mixed tree nut powder composition is described as comprising tree nut flours, it is intended to mean that the tree nut flours are different. For example, if a mixed tree nut powder composition comprises at least a first tree nut flour, a second tree nut flour, and a third tree nut flour, then it is intended and understood that each of the first tree nut flour, the second tree nut flour, and the third tree nut flour are different types of tree nut flour.

In some embodiments, the mixed tree nut powder composition comprises two or more tree nut flours, each selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour.

In some embodiments, the mixed tree nut powder composition comprises three or more tree nut flours each selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour.

In some embodiments, the mixed tree nut powder composition comprises at least a first tree nut flour, a second tree nut flour, and a third tree nut flour, wherein the first tree nut flour is selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour, wherein the second tree nut flour is selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour, and wherein the third tree nut flour is selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour.

In some embodiments, the mixed tree nut powder composition comprises a tree nut flour portion consisting of a first tree nut flour and a second tree nut flour, wherein the first tree nut flour and the second tree nut flour are selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour.

In some embodiments, the mixed tree nut powder composition comprises a tree nut flour portion consisting of a first tree nut flour, a second tree nut flour, and a third tree nut flour, wherein the first tree nut flour, the second tree nut flour, and the third tree nut flour are each selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour.

In some embodiments, the mixed tree nut powder composition comprises at least a first tree nut flour and a second tree nut flour. In some embodiments, the first tree nut flour is selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour. In some embodiments, the second tree nut flour is selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour.

In some embodiments, the mixed tree nut powder composition comprises at least a first tree nut flour, a second tree nut flour, and a third tree nut flour. In some embodiments, the first tree nut flour is selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour. In some embodiments, the second tree nut flour is selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour. In some embodiments, the third tree nut flour selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour.

In some embodiments, the mixed tree nut powder composition comprises at least a first tree nut flour and a second tree nut flour. In some embodiments, the first tree nut flour is walnut flour. In some embodiments, the first tree nut flour is hazelnut flour. In some embodiments, the first tree nut flour is cashew flour. In some embodiments, the second tree nut flour is walnut flour. In some embodiments, the second tree nut flour is hazelnut flour. In some embodiments, the second tree nut flour is cashew flour.

In some embodiments, the mixed tree nut powder composition comprises at least a first tree nut flour, a second tree nut flour, and a third tree nut flour. In some embodiments, the first tree nut flour is walnut flour. In some embodiments, the first tree nut flour is hazelnut flour. In some embodiments, the first tree nut flour is cashew flour. In some embodiments, the second tree nut flour is walnut flour. In some embodiments, the second tree nut flour is hazelnut flour. In some embodiments, the second tree nut flour is cashew flour. In some embodiments, the third tree nut flour is walnut flour. In some embodiments, the third tree nut flour is hazelnut flour. In some embodiments, the third tree nut flour is cashew flour. In some embodiments, the first tree nut flour is hazelnut flour, the second tree nut flour is cashew flour, and the third tree nut flour is walnut flour.

In some embodiments, the mixed tree nut powder composition comprises a tree nut flour portion consisting of a first tree nut flour, a second tree nut flour, and a third tree nut flour. In some embodiments, the first tree nut flour is walnut flour. In some embodiments, the first tree nut flour is hazelnut flour. In some embodiments, the first tree nut flour is cashew flour. In some embodiments, the second tree nut flour is walnut flour. In some embodiments, the second tree nut flour is hazelnut flour. In some embodiments, the second tree nut flour is cashew flour. In some embodiments, the third tree nut flour is walnut flour. In some embodiments, the third tree nut flour is hazelnut flour. In some embodiments, the third tree nut flour is cashew flour. In some embodiments, the first tree nut flour is hazelnut flour, the second tree nut flour is cashew flour, and the third tree nut flour is walnut flour.

### Pharmaceutical Excipients

The mixed tree nut powder compositions described herein include two or more distinct tree nut flours and can be formulated with one or more pharmaceutically acceptable excipients. The one or more excipients can include one or more diluents, one or more lubricants, and/or one or more glidants. In some embodiments, the mixed tree nut powder composition comprises a diluent. In some embodiments, the mixed tree nut powder composition comprises a lubricant. In some embodiments, the mixed tree nut powder composition comprises a glidant. In some embodiments, the mixed tree nut powder composition comprises a diluent and a lubricant. In some embodiments, the mixed tree nut powder composition comprises a diluent, a lubricant, and a glidant. Exemplary diluents include, but are not limited to, pregelatinized starch and microcrystalline cellulose. Exemplary lubricants include, but are not limited to, magnesium stearate. Exemplary glidants include, but are not limited to, colloidal silicon dioxide.

Pharmaceutically acceptable excipients included in the mixed tree nut powder compositions can be selected so that the compositions can be packaged, such as into pharmaceutically acceptable dosage forms (for example, into capsules, tablets, sprinkle capsules, pouches, sachets, or other suitable packaging).

The excipients in the mixed tree nut powder composition can be mixed with multiple tree nut flours simultaneously or in batch (i.e., individually with each tree nut flour). For example, for a mixed tree nut powder composition comprising three types of tree nut flour, the composition can be formed by mixing the first tree nut flour, the second tree nut flour, the third tree nut flour, and the one or more pharmaceutically acceptable excipients simultaneously together. Alternatively, for the mixed tree nut powder composition comprising three types of tree nut flour, each of the first tree nut flour, the second tree nut flour, and the third tree nut flour can be mixed with the one or more pharmaceutically acceptable excipients to form a first mixture, a second mixture, and a third mixture, respectively, and then mixing the first mixture, the second mixture, and the third mixture together. In a further example, to form the mixed tree nut powder composition comprising three distinct tree nut flours, the first tree nut flour, the second tree nut flour, and the third tree nut flour may be mixed together to form a tree nut flour mixture. The tree nut flour mixture may then be mixed with the one or more pharmaceutically acceptable excipients.

The tree nut flours in the mixed tree nut powder composition are generally, but not necessarily, prepared from roasted tree nuts. The tree nuts are also generally, but not necessarily, defatted. Tree nuts may be defatted before roasting, after roasting, or both. In some embodiments of any of the described methods, the first tree nut flour is a defatted tree nut flour. In some embodiments of any of the described methods, the first tree nut flour is a defatted tree nut flour prepared from a roasted tree nut. In some embodiments of any of the described methods, the second tree nut flour is a defatted tree nut flour. In some embodiments of any of the described methods, the second tree nut flour is a defatted tree nut flour prepared from a roasted tree nut. In some embodiments of any of the described methods, the third tree nut flour is a defatted tree nut flour. In some embodiments of any of the described methods, the third tree nut flour is a defatted tree nut flour prepared from a roasted tree nut.

In some embodiments, there is a method of manufacturing a pharmaceutically suitable mixed tree nut powder composition, comprising forming the mixed tree nut powder composition, comprising simultaneously mixing at least a first tree nut flour and a second tree nut flour and one or more pharmaceutically acceptable excipients; assaying the mixed tree nut powder composition by liquid chromatography to determine a ratio for an amount of the first tree nut flour and an amount of the second tree nut flour relative to each other; assessing whether the mixed tree nut powder composition is pharmaceutically suitable, wherein the mixed tree nut powder composition is pharmaceutically suitable only if the ratio is within a predetermined threshold; and packaging the mixed tree nut powder composition if assessed to be pharmaceutically suitable. In some embodiments, the one or more pharmaceutically acceptable excipients comprise a diluent. In some embodiments, the one or more pharmaceutically acceptable excipients comprise a lubricant. In some embodiments, the one or more pharmaceutically acceptable excipients comprise a glidant. In some embodiments, the one or more pharmaceutically acceptable excipients comprise a diluent and a lubricant. In some embodiments, the one or more pharmaceutically acceptable excipients comprise a diluent, a lubricant, and a glidant.

In some embodiments, there is a method of manufacturing a pharmaceutically suitable mixed tree nut powder composition, comprising forming the mixed tree nut powder composition, comprising mixing at least a first tree nut flour and a second tree nut flour to form a tree nut flour mixture, and mixing the tree nut flour mixture with one or more pharmaceutically acceptable excipients; assaying the mixed tree nut powder composition by liquid chromatography to determine a ratio for an amount of the first tree nut flour and an amount of the second tree nut flour relative to each other; assessing whether the mixed tree nut powder composition is pharmaceutically suitable, wherein the mixed tree nut powder composition is pharmaceutically suitable only if the ratio is within a predetermined threshold; and packaging the mixed tree nut powder composition if assessed to be pharmaceutically suitable. In some embodiments, the one or more pharmaceutically acceptable excipients comprise a diluent. In some embodiments, the one or more pharmaceutically acceptable excipients comprise a lubricant. In some embodiments, the one or more pharmaceutically acceptable excipients comprise a glidant. In some embodiments, the one or more pharmaceutically acceptable excipients comprise a diluent and a lubricant. In some embodiments, the one or more pharmaceutically acceptable excipients comprise a diluent, a lubricant, and a glidant.

In some embodiments, there is a method of manufacturing a pharmaceutically suitable mixed tree nut powder composition, comprising forming the mixed tree nut powder composition, comprising mixing a first tree nut flour with one or more pharmaceutically acceptable excipients to form a first mixture, mixing a second tree nut flour with one or more pharmaceutically acceptable excipients to form a second mixture, and mixing the first mixture and the second mixture; assaying the mixed tree nut powder composition by liquid chromatography to determine a ratio for an amount of the first tree nut flour and an amount of the second tree nut flour relative to each other; assessing whether the mixed tree nut powder composition is pharmaceutically suitable, wherein the mixed tree nut powder composition is pharmaceutically suitable only if the ratio is within a predetermined threshold; and packaging the mixed tree nut powder composition if assessed to be pharmaceutically suitable. In some embodiments, the one or more pharmaceutically acceptable excipients comprise a diluent. In some embodiments, the one or more pharmaceutically acceptable excipients comprise a lubricant. In some embodiments, the one or more pharmaceutically acceptable excipients comprise a glidant. In some embodiments, the one or more pharmaceutically acceptable excipients comprise a diluent and a lubricant. In some embodiments, the one or more pharmaceutically acceptable excipients comprise a diluent, a lubricant, and a glidant.

In some embodiments, there is a method of manufacturing a pharmaceutically suitable mixed tree nut powder composition, comprising forming the mixed tree nut powder composition, comprising simultaneously mixing at least a first tree nut flour, a second tree nut flour, a third tree nut flour, and one or more pharmaceutically acceptable excipients; assaying the mixed tree nut powder composition by liquid chromatography to determine a ratio for an amount of the first tree nut flour and an amount of the second tree nut flour relative to each other, determining a second ratio for the amount of the first tree nut flour and the third tree nut flour relative to each other, determining a third ratio for the amount of the second tree nut flour and the third tree nut flour relative to each other; assessing whether the mixed tree nut powder composition is pharmaceutically suitable, wherein the mixed tree nut powder composition is pharmaceutically suitable only if the ratio is within a predetermined threshold, the second ratio is within a second predetermined threshold, and the third ratio is within a third predetermined threshold; and packaging the mixed tree nut powder composition if assessed to be pharmaceutically suitable. In some embodiments, the one or more pharmaceutically acceptable excipients comprise a diluent. In some embodiments, the one or more pharmaceutically acceptable excipients comprise a lubricant. In some embodiments, the one or more pharmaceutically acceptable excipients comprise a glidant. In some embodiments, the one or more pharmaceutically acceptable excipients comprise a diluent and a lubricant. In some embodiments, the one or more pharmaceutically acceptable excipients comprise a diluent, a lubricant, and a glidant.

In some embodiments, there is a method of manufacturing a pharmaceutically suitable mixed tree nut powder composition, comprising forming the mixed tree nut powder composition, comprising mixing at least a first tree nut flour, a second tree nut flour, and a third tree nut flour to form a tree nut flour mixture, and mixing the tree nut flour mixture with one or more pharmaceutically acceptable excipients; assaying the mixed tree nut powder composition by liquid chromatography to determine a ratio for an amount of the first tree nut flour and an amount of the second tree nut flour relative to each other, determining a second ratio for the amount of the first tree nut flour and the third tree nut flour relative to each other, determining a third ratio for the amount of the second tree nut flour and the third tree nut flour relative to each other; assessing whether the mixed tree nut powder composition is pharmaceutically suitable, wherein the mixed tree nut powder composition is pharmaceutically suitable only if the ratio is within a predetermined threshold, the second ratio is within a second predetermined threshold, and the third ratio is within a third predetermined threshold; and packaging the mixed tree nut powder composition if assessed to be pharmaceutically suitable. In some embodiments, the one or more pharmaceutically acceptable excipients comprise a diluent. In some embodiments, the one or more pharmaceutically acceptable excipients comprise a lubricant. In some embodiments, the one or more pharmaceutically acceptable excipients comprise a glidant. In some embodiments, the one or more pharmaceutically acceptable excipients comprise a diluent and a lubricant. In some embodiments, the one or more pharmaceutically acceptable excipients comprise a diluent, a lubricant, and a glidant.

In some embodiments, there is a method of manufacturing a pharmaceutically suitable mixed tree nut powder composition, comprising forming the mixed tree nut powder composition, comprising mixing a first tree nut flour with one or more pharmaceutically acceptable excipients to form a first mixture, mixing a second tree nut flour with one or more pharmaceutically acceptable excipients to form a second mixture, mixing a third tree nut flour with one or more pharmaceutically acceptable excipients to form a third mixture, and mixing the first mixture, the second mixture, and the third mixture; assaying the mixed tree nut powder composition by liquid chromatography to determine a ratio for an amount of the first tree nut flour and an amount of the second tree nut flour relative to each other, determining a second ratio for the amount of the first tree nut flour and the third tree nut flour relative to each other, determining a third ratio for the amount of the second tree nut flour and the third tree nut flour relative to each other; assessing whether the mixed tree nut powder composition is pharmaceutically suitable, wherein the mixed tree nut powder composition is pharmaceutically suitable only if the ratio is within a predetermined threshold, the second ratio is within a second predetermined threshold, and the third ratio is within a third predetermined threshold; and packaging the mixed tree nut powder composition if assessed to be pharmaceutically suitable. In some embodiments, the one or more pharmaceutically acceptable excipients comprise a diluent. In some embodiments, the one or more pharmaceutically acceptable excipients comprise a lubricant. In some embodiments, the one or more pharmaceutically acceptable excipients comprise a glidant. In some embodiments, the one or more pharmaceutically acceptable excipients comprise a diluent and a lubricant. In some embodiments, the one or more pharmaceutically acceptable excipients comprise a diluent, a lubricant, and a glidant.

### Methods of Manufacturing Pharmaceutical Compositions Comprising Tree Nut Flour

Pharmaceutical powder compositions comprising tree nut flour are useful for treating a tree nut allergy by oral immunotherapy (OIT). However, standard methods of processing tree nuts into tree nut flours and in preparing pharmaceutical powder compositions of tree nut flour can change the allergenic activity of the constituent allergenic proteins otherwise present in raw tree nuts. In particular, heating can induce conformational changes in many proteins, which can in some cases be irreversible upon cooling. Thus, in various embodiments, provided herein are methods of manufacturing a pharmaceutical powder composition comprising at least a first type of tree nut flour. These methods are designed to preserve the allergenic activity of the constituent allergenic proteins of the tree nut flour as compared to the allergenic activity of raw tree nuts. These methods are also designed to reduce the moisture content as compared to raw tree nuts, which improves the stability of the tree nut flours.

In some embodiments, there is a method of manufacturing a pharmaceutical powder composition comprising at least a first type of tree nut flour, the method comprising roasting at least a first type of tree nut at a temperature between about 90° C. and about 140° C. (such as between about 90° C. and about 130° C.) to obtain a desired moisture content; forming the at least a first type of tree nut flour using the at least a first type of tree nut after roasting; forming the pharmaceutical powder composition by mixing the at least a first type of tree nut flour with one or more pharmaceutically acceptable excipients; and packaging the pharmaceutical powder composition. In some embodiments, the at least a first type of tree nut flour is cashew flour. In some embodiments, the at least a first type of tree nut flour is hazelnut flour. In some embodiments, the at least a first type of tree nut flour is walnut flour. In some embodiments, the roasting at least a first type of tree nut is at a temperature between about 90° C. and about 140° C. In some embodiments, the roasting at least a first type of tree nut is at a temperature between about 90° C. and about 130° C. In some embodiments, the method comprises removing a portion of fat from the at least a first type of tree nut. In some embodiments, removing a portion of fat comprises pressing. In some embodiments, roasting is for between about 15 minutes to about 90 minutes. In some embodiments, the at least a first type of tree nut has a moisture content of between about 0.5% and about 5% after roasting. In some embodiments, roasting comprises mixing during roasting. In some embodiments, roasting is performed with a multistage continuous nut roaster. In some embodiments, the method further comprises grinding the at least a first type of tree nut.

In some embodiments, there is a method of manufacturing a pharmaceutical powder composition comprising at least a first type of tree nut flour and a second type of tree nut flour, the method comprising roasting at least a first type of tree nut at a temperature between about 90° C. and about 140° C. (such as between about 90° C. and about 130° C.) to obtain a desired moisture content; forming the at least a first type of tree nut flour using the at least a first type of tree nut after roasting; forming the pharmaceutical powder composition by mixing the at least a first type of tree nut flour, the second type of tree nut flour, and one or more pharmaceutically acceptable excipients; and packaging the pharmaceutical powder composition. In some embodiments, the at least a first type of tree nut flour is cashew flour. In some embodiments, the at least a first type of tree nut flour is hazelnut flour. In some embodiments, the at least a first type of tree nut flour is walnut flour. In some embodiments, the at least a second type of tree nut flour is cashew flour. In some embodiments, the at least a second type of tree nut flour is hazelnut flour. In some embodiments, the at least a second type of tree nut flour is walnut flour. In some embodiments, the roasting at least a first type of tree nut is at a temperature between about 90° C. and about 140° C. In some embodiments, the roasting at least a first type of tree nut is at a temperature between about 90° C. and about 130° C. In some embodiments, the method comprises roasting a second type of tree nut at a temperature between about 90° C. and about 140° C. to obtain a desired moisture content and forming the second type of tree nut flour using the second type of tree nut after roasting. In some embodiments, the method comprises roasting a second type of tree nut at a temperature between about 90° C. and about 130° C. to obtain a desired moisture content and forming the second type of tree nut flour using the second type of tree nut after roasting. In some embodiments, the method comprises removing a portion of fat from the at least a first type of tree nut or the second type of tree nut. In some embodiments, removing a portion of fat comprises pressing. In some embodiments, roasting is for between about 15 minutes to about 90 minutes. In some embodiments, the at least a first type of tree nut has a moisture content of between about 0.5% and about 5% after roasting. In some embodiments, the second type of tree nut has a moisture content of between about 0.5% and about 5% after roasting. In some embodiments, roasting comprises mixing during roasting. In some embodiments, roasting is performed with a multistage continuous nut roaster. In some embodiments, the method further comprises grinding the at least a first type of tree nut. In some embodiments, the method further comprises grinding the second type of tree nut.

In some embodiments, there is a method of manufacturing a pharmaceutical powder composition comprising at least a first type of tree nut flour, a second type of tree nut flour, and a third type of tree nut flour, the method comprising roasting at least a first type of tree nut at a temperature between about 90° C. and about 140° C. (such as between about 90° C. and about 130° C.) to obtain a desired moisture content; forming the at least a first type of tree nut flour using the at least a first type of tree nut after roasting; forming the pharmaceutical powder composition by mixing the at least a first type of tree nut flour, the second type of tree nut flour, the third type of tree nut flour, and one or more pharmaceutically acceptable excipients; and packaging the pharmaceutical powder composition. In some embodiments, the at least a first type of tree nut flour is cashew flour. In some embodiments, the at least a first type of tree nut flour is hazelnut flour. In some embodiments, the at least a first type of tree nut flour is walnut flour. In some embodiments, the at least a second type of tree nut flour is cashew flour. In some embodiments, the at least a second type of tree nut flour is hazelnut flour. In some embodiments, the at least a second type of tree nut flour is walnut flour. In some embodiments, the at least a third type of tree nut flour is cashew flour. In some embodiments, the at least a third type of tree nut flour is hazelnut flour. In some embodiments, the at least a third type of tree nut flour is walnut flour. In some embodiments, the roasting at least a first type of tree nut is at a temperature between about 90° C. and about 140° C. In some embodiments, the roasting at least a first type of tree nut is at a temperature between about 90° C. and about 130° C. In some embodiments, the method comprises roasting a second type of tree nut at a temperature between about 90° C. and about 140° C. to obtain a desired moisture content and forming the second type of tree nut flour using the second type of tree nut after roasting. In some embodiments, the method comprises roasting a second type of tree nut at a temperature between about 90° C. and about 130° C. to obtain a desired moisture content and forming the second type of tree nut flour using the second type of tree nut after roasting. In some embodiments, the method comprises roasting a third type of tree nut at a temperature between about 90° C. and about 140° C. to obtain a desired moisture content and forming the third type of tree nut flour using the third type of tree nut after roasting. In some embodiments, the method comprises roasting a third type of tree nut at a temperature between about 90° C. and about 130° C. to obtain a desired moisture content and forming the third type of tree nut flour using the third type of tree nut after roasting. In some embodiments, the method comprises removing a portion of fat from the at least a first type of tree nut, the second type of tree nut, or the third type of tree nut. In some embodiments, removing a portion of fat comprises pressing. In some embodiments, roasting is for between about 15 minutes to about 90 minutes. In some embodiments, the at least a first type of tree nut has a moisture content of between about 0.5% and about 5% after roasting. In some embodiments, the second type of tree nut has a moisture content of between about 0.5% and about 5% after roasting. In some embodiments, third type of tree nut has a moisture content of between about 0.5% and about 5% after roasting. In some embodiments, roasting comprises mixing during roasting. In some embodiments, roasting is performed with a multistage continuous nut roaster. In some embodiments, the method further comprises grinding the at least a first type of tree nut. In some embodiments, the method further comprises grinding the second type of tree nut. In some embodiments, the method further comprises grinding the third type of tree nut.

### Tree Nut Flours

The pharmaceutical powder compositions comprise at least a first type of tree nut flour, but may comprise two types of tree nut flour, three types or tree nut flour, four types of tree nut flour, or more. The types of tree nut flours may include, but are not limited to, walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour. The tree nut flours may be formed from any type of tree nut including, but not limited to, walnuts, almonds, pistachios, cashews, pecans, hazelnuts, macadamias, Brazil nuts, pine nuts, beechnuts, butternuts, chestnuts, chinquapins, coconuts, ginkgo nuts, hickory nuts, lichee nuts, pili nuts, and sheanuts.

In some embodiments, the pharmaceutical powder composition comprises at least a first type of tree nut flour selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour.

In some embodiments, the pharmaceutical powder composition comprises at least a first type of tree nut flour and a second type of tree nut flour each selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour.

In some embodiments, the pharmaceutical powder composition comprises at least a first type of tree nut flour, a second type of tree nut flour, and a third type of tree nut flour each selected from the list consisting of walnut flour, almond flour, pistachio flour, cashew flour, pecan flour, hazelnut flour, macadamia flour, Brazil nut flour, pine nut flour, beechnut flour, butternut flour, chestnut flour, chinquapin flour, coconut flour, ginkgo nut flour, hickory nut flour, lichee nut flour, pili nut flour, and sheanut flour.

In some embodiments, the pharmaceutical powder composition comprises at least a first type of tree nut flour, wherein the first type of tree nut flour is walnut flour. In some embodiments, the pharmaceutical powder composition comprises at least a first type of tree nut flour, wherein the first type of tree nut flour is cashew flour. In some embodiments, the pharmaceutical powder composition comprises at least a first type of tree nut flour, wherein the first type of tree nut flour is hazelnut flour.

In some embodiments, the pharmaceutical powder composition comprises at least a first type of tree nut flour and a second type of tree nut flour. In some embodiments, the first type of tree nut flour is hazelnut flour. In some embodiments, the first type of tree nut flour is cashew flour. In some embodiments, the first type of tree nut flour is walnut flour. In some embodiments, the second type of tree nut flour is hazelnut flour. In some embodiments, the second type of tree nut flour is cashew flour. In some embodiments, the second type of tree nut flour is walnut flour.

In some embodiments, the pharmaceutical powder composition comprises at least a first type of tree nut flour, a second type of tree nut flour, and a third type of tree nut flour. In some embodiments, the first type of tree nut flour is hazelnut flour. In some embodiments, the first type of tree nut flour is cashew flour. In some embodiments, the first type of tree nut flour is walnut flour. In some embodiments, the second type of tree nut flour is hazelnut flour. In some embodiments, the second type of tree nut flour is cashew flour. In some embodiments, the second type of tree nut flour is walnut flour. In some embodiments, the third type of tree nut flour is hazelnut flour. In some embodiments, the third type of tree nut flour is cashew flour. In some embodiments, the third type of tree nut flour is walnut flour.

The tree nut flours in the pharmaceutical powder composition are generally, but not necessarily, defatted. Tree nuts may be defatted before roasting, after roasting, or both. In some embodiments of any of the described methods, the first type of tree nut flour is a defatted tree nut flour. In some embodiments of any of the described methods, the first type of tree nut flour is a defatted tree nut flour prepared from a roasted tree nut. In some embodiments of any of the described methods, the second type of tree nut flour is a defatted tree nut flour. In some embodiments of any of the described methods, the second type of tree nut flour is a defatted tree nut flour prepared from a roasted tree nut. In some embodiments of any of the described methods, the third type of tree nut flour is a defatted tree nut flour. In some embodiments of any of the described methods, the third type of tree nut flour is a defatted tree nut flour prepared from a roasted tree nut.

### Roasting

The methods of manufacturing a pharmaceutical powder composition comprising at least a first type of tree nut flour described herein comprise a step of roasting tree nuts. The roasting step functions, in part, to reduce the moisture content of the tree nuts, which improves the stability of the compositions. It has been found that roasting tree nuts at a lower temperature than conventionally used to roast tree nuts can maintain the protein and allergenic profile of the tree nuts after roasting (and thus the resulting tree nut flours) as compared to higher temperature roasting. Roasting at temperatures above 140° C. (such as 150° C.) can significantly modify the protein profile of tree nut flours as compared to tree nuts before roasting. Proteins that are damaged, such as by denaturing, may no longer be recognized by the immune system, and therefore may not be effective at desensitizing patients to tree nut allergy as part of an OIT regimen.

In some embodiments, roasting a type of tree nut is at a temperature between about 90° C. and about 140° C., such as about 90° C. to about 130° C., about 90° C. to about 120° C., about 90° C. to about 110° C., about 90° C. to about 100° C., about 100° C. to about 140° C., about 100° C. to about 130° C., about 100° C. to about 120° C., about 100° C. to about 110° C., about 110° C. to about 140° C., about 110° C. to about 130° C., or about 110° C. to about 120° C. In some embodiments, roasting a type of tree nut is at a temperature between about 90° C. to about 140° C. In some embodiments, roasting a type of tree nut is at a temperature between about 90° C. to about 130° C. In some embodiments, roasting a type of tree nut is at a temperature between about 90° C. to about 120° C. In some embodiments, roasting a type of tree nut is at a temperature between about 100° C. to about 130° C.

The tree nuts may be mixed or agitated while they are being roasted. The agitation or mixing allows for more even roasting and more consistent heating throughout the batch. The type of roasting device, the tree nut bed depth, and the use of mixing or agitation can be adjusted to ensure the roasting temperature is maintained within the appropriate range throughout the tree nut batch and to achieve a consistent reduction in moisture content throughout the batch. To achieve this consistency, a batch of tree nuts can be split into subbatches and each sub-batch can move through different roasting stages, for example, in a multistage continuous roasting device. Each stage of the device can have a different temperature (as long as no stage causes the tree nuts to exceed the maximum temperature).

The tree nuts should be roasted within the appropriate temperature range for sufficient time to achieve the desired moisture content. In some embodiments, the desired moisture content is between about 0.4% and about 4% after roasting, such as about 0.4% to about 4%, about 0.4% to about 3%, about 0.4% to about 2%, about 1% to about 4%, about 1% to about 3%, about 2% to about 4%, or about 3% to about 4% after roasting. In some embodiments, the desired moisture content is between about 0.4% to about 4%. In some embodiments, the desired moisture content is between about 0.4% to about 2%. In some embodiments, the desired moisture content is between about 0.4% to about 1.5%.

Tree nuts are used to form tree nut flours for the pharmaceutical powder compositions. To form the tree nut flours, in addition to roasting the tree nuts, additional processing steps can be performed, including, but not limited to, pasteurization, grinding, defatting, and pressing.

In some embodiments, forming the tree nut flour comprises pasteurizing tree nuts. In some embodiments, forming the tree nut flour comprises grinding roasted tree nuts. The tree nuts are ground to achieve a particular particle size profile. In some embodiments, the ground tree nuts have a median particle size less than about 100 μm. In some embodiments, the ground tree nuts have a Dv(90) particle size less than about 300 μm.

In some embodiments, forming the tree nut flour comprises defatting a portion of fat from ground tree nuts. In some embodiments, defatting a portion of fat from ground tree nuts comprises pressing ground tree nuts. In some embodiments, defatting a portion of fat from ground tree nuts comprises pressing ground tree nuts into a pressed cake. In some embodiments, defatting a portion of fat from ground tree nuts comprises pressing ground tree nuts into a press cake and processing the ground cake to form the tree nut flour.

### Pharmaceutical Excipients

The tree nut flours can be mixed with one or more pharmaceutically acceptable excipients to form the pharmaceutical powder compositions. The one or more pharmaceutically acceptable excipients can include one or more diluents, one or more lubricants, and/or one or more glidants. In some embodiments, the pharmaceutical powder composition comprises a diluent. In some embodiments, the pharmaceutical powder composition comprises a lubricant. In some embodiments, the pharmaceutical powder composition comprises a glidant. In some embodiments, the pharmaceutical powder composition comprises a diluent and a lubricant. In some embodiments, the pharmaceutical powder composition comprises a diluent, a lubricant, and a glidant. Exemplary diluents include, but are not limited to, pregelatinized starch and microcrystalline cellulose. Exemplary lubricants include, but are not limited to, magnesium stearate. Exemplary glidants include, but are not limited to, colloidal silicon dioxide.

Pharmaceutically acceptable excipients included in the mixed tree nut powder compositions can be selected so that the compositions can be packaged, such as into pharmaceutically acceptable dosage forms (for example, into capsules, sachets, or other suitable packaging).

Methods of Making Mixed Nut Compositions with Quality Control

The methods of manufacturing a pharmaceutically suitable mixed tree nut powder composition include a quality control process as described in a preceding section of this application. The tree nut flours in the composition are formed from tree nuts. Standard methods of processing tree nuts into tree nut flours can change the allergenic activity of the constituent allergenic proteins otherwise present in raw tree nuts. In particular, heating can induce conformation changes in many proteins, which can in some cases be irreversible upon cooling. Described herein are methods of manufacturing a pharmaceutically suitable mixed tree nut powder composition which includes a quality control process and forming one or more tree nut flours from one or more types of tree nuts.

In some embodiments, there is a method of manufacturing a pharmaceutically suitable mixed tree nut powder composition, comprising roasting a first type of tree nut at a temperature between about 90° C. and about 140° C. (such as between about 90° C. and about 130° C.) to obtain a desired moisture content; forming a first tree nut flour using the first type of tree nut after roasting; forming the mixed tree nut powder composition, comprising mixing at least the first tree nut flour and a second tree nut flour; assaying the mixed tree nut powder composition by liquid chromatography to determine a ratio for an amount of the first tree nut flour and an amount of the second tree nut flour relative to each other; assessing whether the mixed tree nut powder composition is pharmaceutically suitable, wherein the mixed tree nut powder composition is pharmaceutically suitable only if the ratio is within a predetermined threshold; and packaging the mixed tree nut powder composition if assessed to be pharmaceutically suitable. In some embodiments, the method comprises roasting a second type of tree nut at a temperature between about 90° C. and about 140° C. (such as between about 90° C. and about 130° C.) to obtain a desired moisture content and forming the second tree nut flour using the second type of tree nut after roasting. In some embodiments, the first type of tree nut is cashew. In some embodiments, the first type of tree nut is hazelnut. In some embodiments, the first type of tree nut is walnut. In some embodiments, the second type of tree nut is cashew. In some embodiments, the second type of tree nut is hazelnut. In some embodiments, the second type of tree nut is walnut. In some embodiments, the roasting a first type of tree nut is at a temperature between about 90° C. and about 140° C. In some embodiments, the roasting a first type of tree nut is at a temperature between about 90° C. and about 130° C. In some embodiments, the method comprises removing a portion of fat from the first type of tree nut. In some embodiments, removing a portion of fat comprises pressing. In some embodiments, roasting is for about 15 minutes to about 90 minutes. In some embodiments, the first type of tree nut has a moisture content of between about 0.5% and about 5% after roasting. In some embodiments, the second type of tree nut has a moisture content of between about 0.5% and about 5% after roasting. In some embodiments, roasting comprises mixing during roasting. In some embodiments, roasting is performed with a multistage continuous nut roaster. In some embodiments, the method further comprises grinding the first type of tree nut. In some embodiments, the method further comprises grinding the second type of tree nut.

In some embodiments, there is a method of manufacturing a pharmaceutically suitable mixed tree nut powder composition, comprising roasting a first type of tree nut at a temperature between about 90° C. and about 140° C. (such as between about 90° C. and about 130° C.) to obtain a desired moisture content; forming a first tree nut flour using the first type of tree nut after roasting; forming the mixed tree nut powder composition, comprising mixing at least the first tree nut flour, a second tree nut flour, and a third tree nut flour; assaying the mixed tree nut powder composition by liquid chromatography to determine a ratio for an amount of the first tree nut flour and an amount of the second tree nut flour relative to each other, determining a second ratio for the amount of the first tree nut flour and an amount for the third tree nut flour relative to each other, determining a third ratio for the amount of the second tree nut flour and an amount for the third tree nut flour relative to each other; assessing whether the mixed tree nut powder composition is pharmaceutically suitable, wherein the mixed tree nut powder composition is pharmaceutically suitable only if the ratio is within a predetermined threshold, the second ratio is within a second predetermined threshold, and the third ratio is within a third predetermined threshold; and packaging the mixed tree nut powder composition if assessed to be pharmaceutically suitable. In some embodiments, the method comprises roasting a second type of tree nut at a temperature between about 90° C. and about 130° C. to obtain a desired moisture content and forming the second tree nut flour using the second type of tree nut after roasting. In some embodiments, the method comprises roasting a second type of tree nut at a temperature between about 90° C. and about 140° C. to obtain a desired moisture content and forming the second tree nut flour using the second type of tree nut after roasting. In some embodiments, the method comprises roasting a third type of tree nut at a temperature between about 90° C. and about 130° C. to obtain a desired moisture content and forming the third tree nut flour using the third type of tree nut after roasting. In some embodiments, the method comprises roasting a third type of tree nut at a temperature between about 90° C. and about 140° C. to obtain a desired moisture content and forming the third tree nut flour using the third type of tree nut after roasting. In some embodiments, the first type of tree nut is cashew. In some embodiments, the first type of tree nut is hazelnut. In some embodiments, the first type of tree nut is walnut. In some embodiments, the second type of tree nut is cashew. In some embodiments, the second type of tree nut is hazelnut. In some embodiments, the second type of tree nut is walnut. In some embodiments, the third type of tree nut is cashew. In some embodiments, the third type of tree nut is hazelnut. In some embodiments, the third type of tree nut is walnut. In some embodiments, the roasting a first type of tree nut is at a temperature between about 90° C. and about 140° C. In some embodiments, the roasting a first type of tree nut is at a temperature between about 90° C. and about 130° C. In some embodiments, the method comprises removing a portion of fat from the first type of tree nut. In some embodiments, removing a portion of fat comprises pressing. In some embodiments, roasting is for about 15 minutes to about 90 minutes. In some embodiments, the first type of tree nut has a moisture content of between about 0.5% and about 5% after roasting. In some embodiments, the second type of tree nut has a moisture content of between about 0.5% and about 5% after roasting. In some embodiments, the third type of tree nut has a moisture content of between about 0.5% and about 5% after roasting. In some embodiments, roasting comprises mixing during roasting. In some embodiments, roasting is performed with a multistage continuous nut roaster. In some embodiments, the method further comprises grinding the first type of tree nut. In some embodiments, the method further comprises grinding the second type of tree nut. In some embodiments, the method further comprises grinding the third type of tree nut.

### Roasting

The methods of manufacturing a mixed tree nut powder composition including the quality control process may comprise a step of roasting tree nuts to form tree nut flour. The roasting step functions, in part, to reduce moisture content of the tree nuts, which improves the stability of the compositions. Roasting also functions to reduce the bioburden of the tree nuts. It has been found that roasting tree nuts at a lower temperature than conventionally used to roast tree nuts can maintain the protein and allergenic profile of the tree nuts after roasting (and thus the resulting tree nut flours) as compared to higher temperature roasting. Roasting at temperatures above 140° C. (such as 150° C.) can significantly modify the protein profile of tree nut flours as compared to tree nuts before roasting. Proteins that are damaged, such as by denaturing, may no longer be recognized by the immune system, and therefore may not be effective at desensitizing patients to tree nut allergy as part of an OIT regimen.

In some embodiments, roasting a type of tree nut is at a temperature between about 90° C. and about 140° C., such as about 90° C. to about 130° C., about 90° C. to about 120° C., about 90° C. to about 110° C., about 90° C. to about 100° C., about 100° C. to about 140° C., about 100° C. to about 130° C., about 100° C. to about 120° C., about 100° C. to about 110° C., about 110° C. to about 140° C., about 110° C. to about 130° C., or about 110° C. to about 120° C. In some embodiments, roasting a type of tree nut is at a temperature between about 90° C. to about 140° C. In some embodiments, roasting a type of tree nut is at a temperature between about 90° C. to about 130° C. In some embodiments, roasting a type of tree nut is at a temperature between about 90° C. to about 120° C. In some embodiments, roasting a type of tree nut is at a temperature between about 100° C. to about 130° C.

The roasting time and temperature are affected by certain other operational parameters, including the depth of the tree nuts, the air flow rate, and the air distribution. The temperature at the middle/center of the tree nut bed/batch typically increases at the slowest rate. Thus, in some embodiments, the roasting is carried out with agitation or mixing. The agitation or mixing allows for more even roasting and more consistent heating throughout the batch. It is understood that any particular temperature range, such as about 90° C. to about 130° C., means that the tree nuts are held at between about 90° C. to about 130° C. for at least one period of time and that the tree nuts should not significantly exceed 130° C. The type of roasting device, the tree nut bed depth, and the use of mixing or agitation can be optimized to ensure the roasting temperature is maintained within the appropriate range throughout the tree nut batch and to achieve a consistent reduction in moisture content throughout the batch. To achieve this consistency, a batch of tree nuts can be split into subbatches and each subbatch can move through different roasting stages, for example, in a multistage continuous roasting device. Each stage of the device can have a different temperature (as long as no stage causes the tree nuts to exceed the maximum temperature).

The tree nuts should be roasted within the appropriate temperature range for sufficient time to achieve the desired moisture content. In some embodiments, the desired moisture content is between about 0.5% and about 7% after roasting, such as about 0.5% to about 6%, about 0.5% to about 5%, about 0.5% to about 4%, about 0.5% to about 3%, about 0.5% to about 2%, about 1% to about 7%, about 1% to about 6%, about 1% to about 5%, about 1% to about 4%, about 1% to about 3%, about 2% to about 7%, about 2% to about 6%, about 2% to about 5%, about 2% to about 4%, about 3% to about 7%, about 3% to about 6%, about 3% to about 5%, about 4% to about 7%, or about 4% to about 6% after roasting. In some embodiments, the desired moisture content is between about 0.5% to about 5%.

### Other Processing Steps in Forming the Tree Nut Flours

Tree nuts are used to form tree nut flours for the mixed tree nut powder compositions. To form the tree nut flours, in addition to roasting the tree nuts, additional processing steps can be performed, including, but not limited to, pasteurization, grinding, defatting, and pressing.

In some embodiments, forming the tree nut flour comprises pasteurizing tree nuts. In some embodiments, forming the tree nut flour comprises grinding roasted tree nuts. The tree nuts are ground to achieve a particular particle size profile. In some embodiments, the ground tree nuts have a median particle size less than about 100 μm. In some embodiments, the ground tree nuts have a Dv(90) particle size less than about 300 μm.

In some embodiments, forming the tree nut flour comprises defatting a portion of fat from ground tree nuts. In some embodiments, defatting a portion of fat from ground tree nuts comprises pressing ground tree nuts. In some embodiments, defatting a portion of fat from ground tree nuts comprises pressing ground tree nuts into a pressed cake. In some embodiments, defatting a portion of fat from ground tree nuts comprises pressing ground tree nuts into a press cake and processing the ground cake to form the tree nut flour.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1. Method of Assessing Proportions of Tree Nut Flours in a Mixed Tree Nut Powder Composition

This example demonstrates a method of determining the proportion of individual tree nut flours relative to each other within a mixed tree nut powder composition.

Defatted, roasted tree nut flours (walnut flour, hazelnut flour, and cashew flour) were combined to form a “mixed tree nut powder composition.”

Ultra-Performance Liquid Chromatography (UPLC) analysis was conducted using a C18 Poroshell 300SB column and Agilent Poroshell 300SB-C18 guard column.

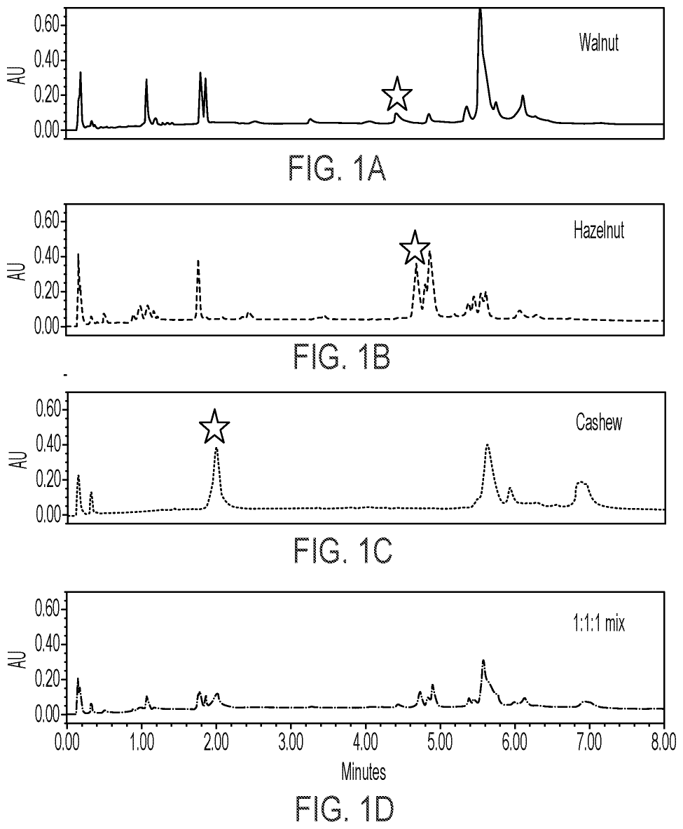

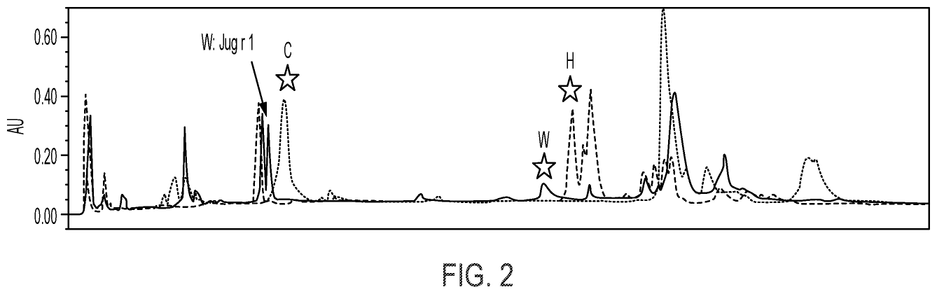

The mixed tree nut powder composition and each individual tree nut flour (walnut flour, hazelnut flour, and cashew flour) were analyzed by UPLC (FIGS. 1A-1D). Peaks were identified for each individual tree nut flour chromatogram. The individual nut flour chromatograms were overlaid and annotated as shown in FIG. 2. Peaks with resolutions equal to or greater than 0.6 as calculated by USP29 method are annotated for cashew (C), walnut (W), and hazelnut (H) flours.

Peaks were identified for each individual tree nut flour by LC-MS. For hazelnut, peaks corresponding to Cor a 1, Cor a 8, Cor a 14, Cor a 9, Cor a 11 were identified. For walnut, peaks corresponding to Jug r 1, Jug r 4, and Jug r 5 were identified. For cashew, peaks corresponding to Ana o 3 and Ana o 2 were identified.

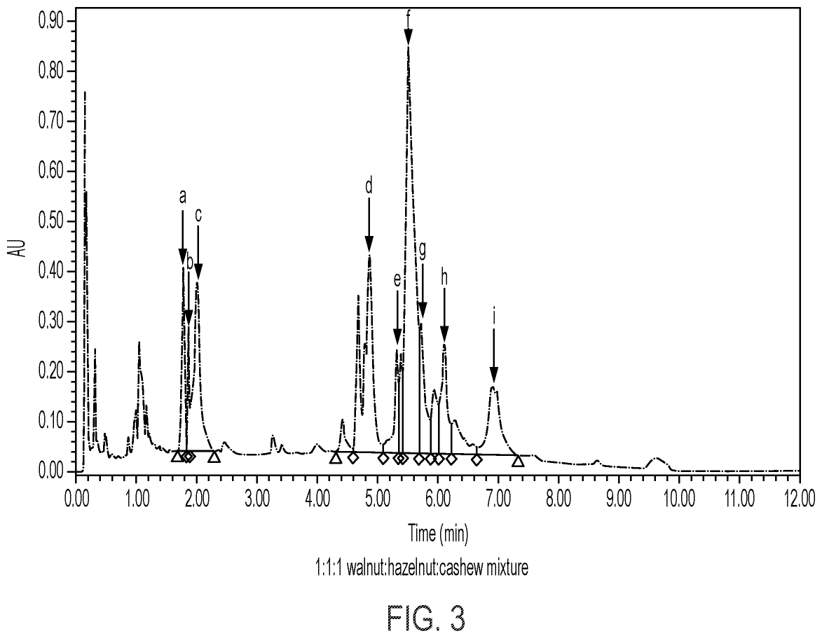

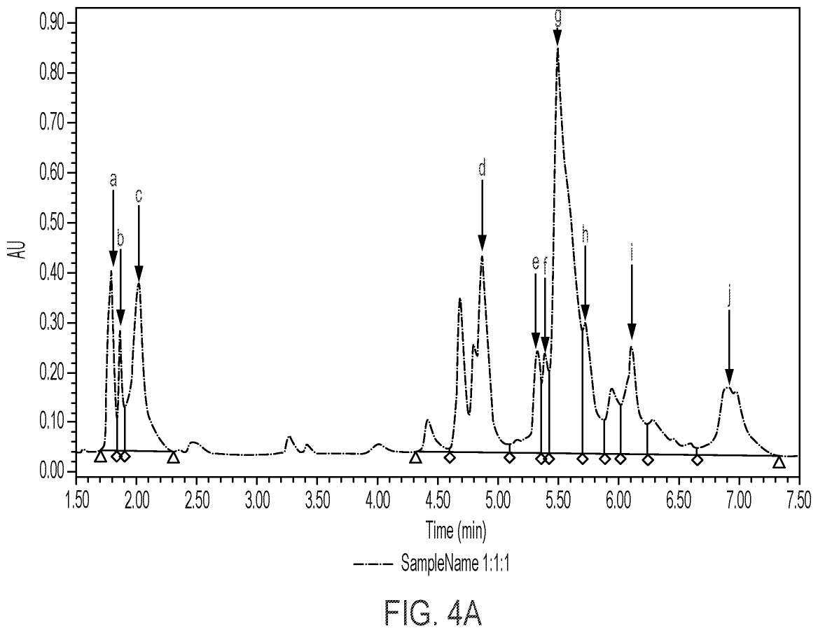

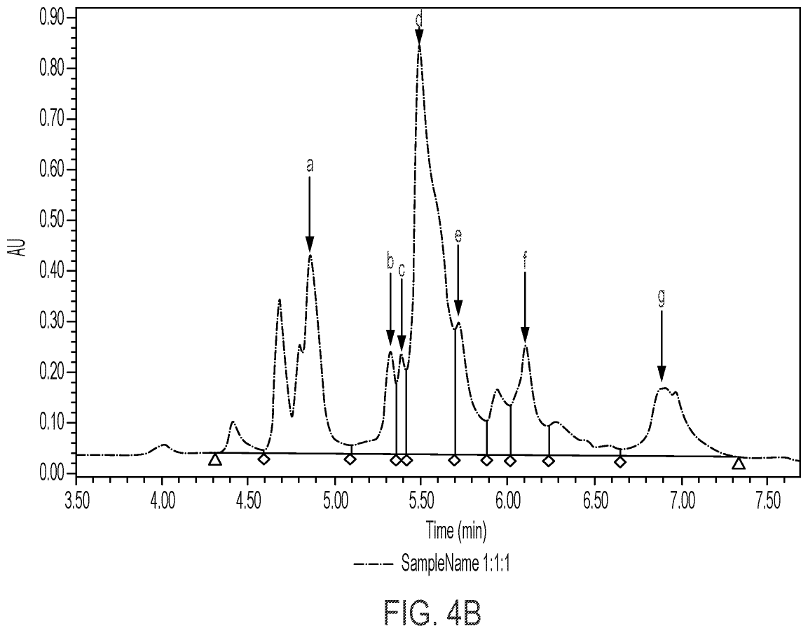

In the 1:1:1 mixture of cashew, walnut, and hazelnut flours, peaks in the mixture were identified. The RP-UPLC chromatogram of the mixture with identified peaks is shown in FIG. 3. Zoomed views of the chromatogram are shown in FIG. 4A and FIG. 4B with annotated peaks.

In a separate experiment, peaks in the 1:1:1 mixture were identified and assessed for resolution by the USP29 method. The results are summarized in Table 1, below.

TABLE 1
Resolution of peaks in mixed tree nut flour mixture.

| Identified Protein | Retention time | USP Resolution |
|---|---|---|
| Cor a 14 | 2.059 | 4.9 |
| Jug r 1 | 2.131 | 0.4 |
| Ana o 3 | 2.277 | 0.6 |
| Jug r 3 | 2.735 | 1.0 |
| Jug r 2 | 4.631 | 3.9 |
| Cor a 9 peak 1 | 4.839 | 0.9 |
| Cor a 9 peak 2 | 5.007 | 0.8 |
| Cor a 11 | 6.418 | 0.4 |
| Ana 0 2 peak B | 7.066 | 0.8 |

The ability of this method to determine the ratio of each individual tree nut flour in the mixed tree nut powder composition was assessed by mixing the three individual tree nut flours together at different ratios to each other and assessing the change in integrated peak area by UPLC of the respective tree nut flours. The control sample was a 1:1:1 mixture of cashew, walnut, and hazelnut flours. 0.5:1:1, 1:0.5:1, and 1:1:0.5 mixtures were prepared of cashew, walnut, and hazelnut flours to create a half cashew mixture, a half walnut mixture, and a half hazelnut mixture, respectively. Based on the resolutions summarized in Table 1, above, Jug r 2, Cor a 9, and Ana o 3 were selected as sufficiently resolved quantitative peaks. Integrating these peaks, the percent compared to control was calculated for each tree nut flour in the mixtures. The results are summarized in Table 2, below.

TABLE 2
Peak area for tree nut flour mixtures.

| | | |
|---|---|---|
| | Absolute peak area | Percent compared |
| Tree Nut Flour | (A.U.) | to control (%) |

1:1:1 Control

| | | |
|---|---|---|
| Cashew | 603186 | 100 |
| Walnut | 94986 | 100 |
| Hazelnut | 1018426 | 100 |

Half Cashew

| | | |
|---|---|---|
| Cashew | 341482 | 57 |
| Walnut | 92918 | 98 |
| Hazelnut | 1066074 | 105 |

Half Walnut

| | | |
|---|---|---|
| Cashew | 659641 | 109 |
| Walnut | 49916 | 53 |
| Hazelnut | 1054609 | 104 |

Half Hazelnut

| | | |
|---|---|---|
| Cashew | 637252 | 106 |
| Walnut | 91174 | 96 |
| Hazelnut | 523426 | 51 |

As shown in Table 2, even though each sample was complicated by the presence of three distinct types of tree nut flours, the identification of unique peaks corresponding to each individual tree nut flour allowed for accurate quantification of the proportion of each tree nut flour within the mixture, as confirmed by varying the proportion of cashew, walnut, or hazelnut flour in the mixture. For example, in the half cashew mixture, walnut and hazelnut were accurately assessed (98% and 105% of control, respectively), while the cashew proportion was assessed as approximately halved (57% of control).

### Example 2. Effect of Roasting Time and Temperature of Defatted Tree Nut Powders

To control bioburden and reduce moisture to maintain stability, tree nuts can be roasted prior to forming tree nut flours. This example demonstrates methods of preparing tree nut flours.

Walnut, hazelnut, and cashew tree nuts were roasted at a temperature between about 150° C. and about 160° C. Roasting reduced moisture content of walnuts from about 3.1% to about 0.7%, of cashews from about 4.6% to about 0.7%, and of hazelnuts from about 3.6% to about 0.7%. SDS-PAGE and Western blot analysis were performed after roasting to determine the change in protein content for each tree nut. The results are summarized in Table 3, below.

TABLE 3
Characteristics of walnut, cashew, and hazelnut powders.

| | | Protein | | |
|---|---|---|---|---|
| Product (powder) | Fat (%) | (N × 6.25)(%) | ×50 (μm) | ×90(μm) |
| Walnut | 10.7 | 52.5 | 12.0 | 71 |
| Cashew | 11.4 | 37.7 | 7.5 | 34 |
| Hazelnut | 18.6 | 47.4 | 13.1 | 57 |

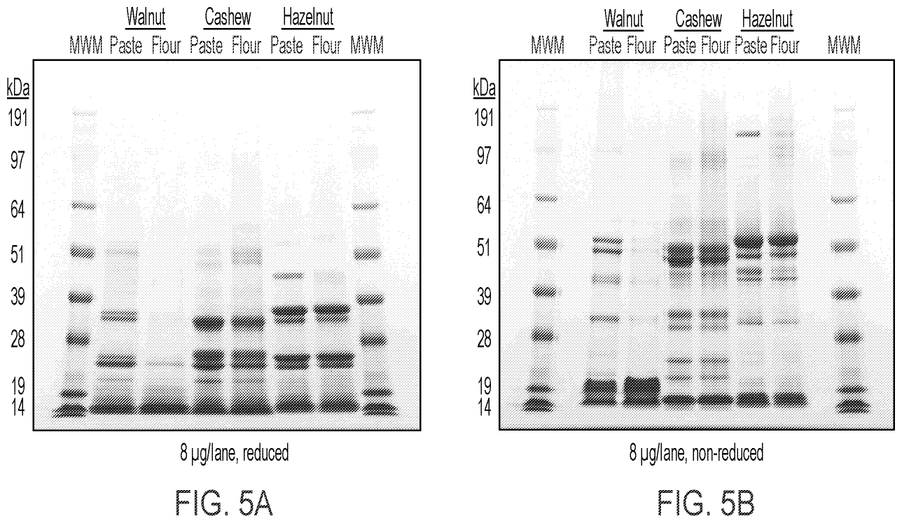

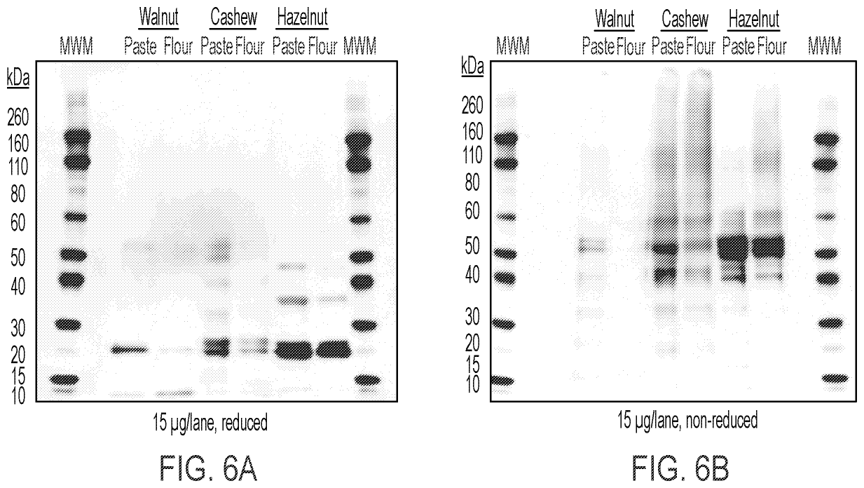

The protein contents were compared by SDS-PAGE and Western blot before and after roasting. SDS-PAGE results for cashew, hazelnut, and walnut flours before and after roasting, and under reducing and non-reducing conditions, are shown in FIGS. 5A and 5B. Western blot results for cashew, hazelnut, and walnut flours before and after roasting, and under reducing and non-reducing conditions, are shown in FIGS. 6A and 6B. In FIGS. 5A, 5B, 6A, and 6B, paste is before roasting, and flour is after roasting. The primary antibody for Western blot analysis was an allergic plasma pool in PBST. The secondary antibody was goat IgG anti-human-IgE conjugated to HRP. As shown by both SDS-PAGE and Western blot analysis, the protein profile of walnut was most substantially changed by roasting between about 150° C. and 160° C.

To assess whether lower-temperature roasting could preserve the allergen content of walnuts while achieving an acceptable moisture content, walnuts were roasted at about 90° C., about 110° C., and about 130° C. Walnuts were roasted at the temperature and durations indicated in Table 4, below, and then allowed to cool to room temperature. After cooling, roasted walnuts were extracted and clarified by centrifugation at 4000×g for 20 minutes. Supernatants were removed for Bradford assay to determine protein concentration and for analysis by SDS-PAGE and Western blot.

TABLE 4
Summary of roasting time and temperature for walnuts

| | 90° C. | 110° C. | 130° C. |
|---|---|---|---|
| Roasting time | 15 | 15 | 15 |
| (minutes) | 30 | 30 | 30 |
| | 45 | — | |

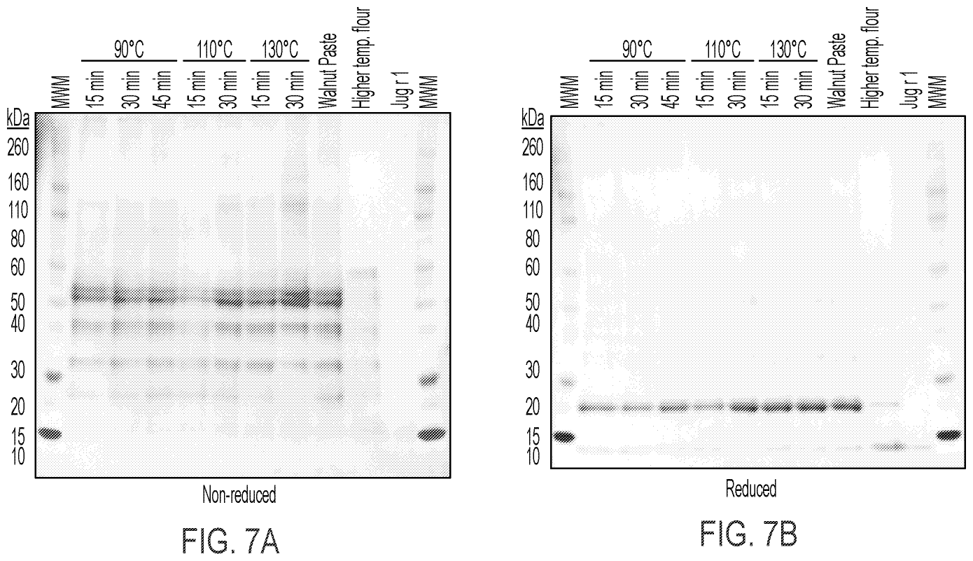

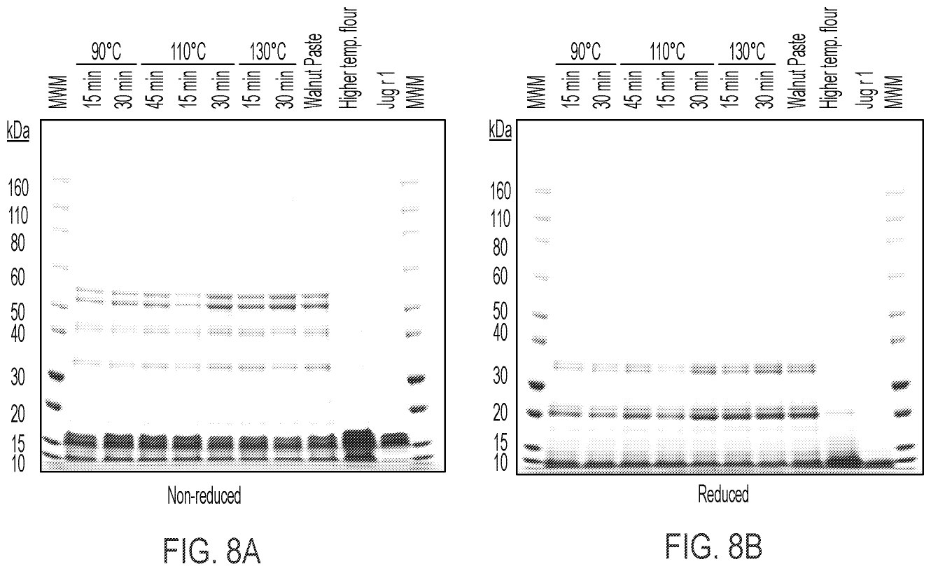

SDS-PAGE and Western blot analyses were performed on the roasted walnut samples after extraction with reducing and non-reducing conditions. The primary antibody for Western blot analysis was an allergic plasma pool in PBST. The secondary antibody was goat IgG anti-human-IgE conjugated to HRP. FIGS. 7A and 7B show the non-reduced and reduced Western blot analysis of walnuts before and after roasting at the lower temperatures. FIGS. 8A and 8B show the non-reduced and reduced SDS-PAGE analysis for walnuts before and after roasting at the lower temperatures. Walnut paste show the walnuts before roasting. The walnut flour with the higher temperature roasting is shown for comparison (higher temperature flour). A jug r 1 control was also loaded. As illustrated by FIGS. 7A, 7B, 8A, and 8B, the lower-temperature roasting conditions produced protein profiles very similar to the unroasted paste, whereas the higher-temperature roasting condition yielded a very different protein profile.

1. A method of assessing a mixed tree nut powder composition for pharmaceutical suitability, wherein the mixed tree nut powder composition comprises at least a first tree nut flour and a second tree nut flour, comprising:
assaying the mixed tree nut powder composition by liquid chromatography to determine a ratio for an amount of the first tree nut flour and an amount of the second tree nut flour relative to each other; and
assessing whether the mixed tree nut powder composition is pharmaceutically suitable, wherein the mixed tree nut powder composition is pharmaceutically suitable only if the ratio is within a predetermined threshold.

2. The method of claim 1, wherein the mixed tree nut powder composition further comprises at least a third tree nut flour, wherein assaying the mixed tree nut powder composition by liquid chromatography further comprises:
determining a second ratio for the amount of the first tree nut flour and the third tree nut flour relative to each other; and
determining a third ratio for the amount of the second tree nut flour and the third tree nut flour relative to each other;
wherein the mixed tree nut powder composition is pharmaceutically suitable only if the second ratio is within a second predetermined threshold and the third ratio is within a third predetermined threshold.

3. The method of claim 1, wherein the mixed tree nut powder composition is assessed as pharmaceutically suitable and the method further comprises packaging the mixed tree nut powder composition.

4. (canceled)

5. The method of claim 1, wherein the mixed tree nut powder composition is assessed as pharmaceutically unsuitable, wherein the mixed tree nut powder composition is pharmaceutically unsuitable if the ratio is not within a predetermined threshold, the method further comprising discarding the mixed tree nut powder composition.

6-7. (canceled)

8. A method of manufacturing a pharmaceutically suitable mixed tree nut powder composition, comprising:
forming the mixed tree nut powder composition, comprising mixing at least a first tree nut flour and a second tree nut flour;
assaying the mixed tree nut powder composition by liquid chromatography to determine a ratio for an amount of the first tree nut flour and an amount of the second tree nut flour relative to each other;
assessing whether the mixed tree nut powder composition is pharmaceutically suitable, wherein the mixed tree nut powder composition is pharmaceutically suitable only if the ratio is within a predetermined threshold;
packaging the mixed tree nut powder composition if assessed to be pharmaceutically suitable.

9. The method of claim 8, wherein forming the mixed tree nut powder composition further comprises mixing a third tree nut flour with the first tree nut flour and the second tree nut flour; wherein assaying the mixed tree nut powder composition by liquid chromatography further comprises:
determining a second ratio for the amount of the first tree nut flour and the third tree nut flour relative to each other; and
determining a third ratio for the amount of the second tree nut flour and the third tree nut flour relative to each other;
wherein the mixed tree nut powder composition is pharmaceutically suitable only if the second ratio is within a second predetermined threshold and the third ratio is within a third predetermined threshold.

10. The method of claim 8, wherein the mixed tree nut powder composition is packaged in a pharmaceutically acceptable dosage form.

11. The method of claim 1, wherein the liquid chromatography is reversed-phase ultra-high-performance liquid chromatography (RP-UPLC).

12. The method of claim 1, wherein the predetermined threshold is a range set between about 0.75 and about 1.25.

13-20. (canceled)

21. The method of claim 8, wherein forming the mixed tree nut powder composition comprises:
mixing the first tree nut flour with a first amount of the one or more pharmaceutically acceptable excipients to form a first mixture;
mixing the second tree nut flour with a second amount of the one or more pharmaceutically acceptable excipients to form a second mixture;
mixing the third tree nut flour with a third amount of the one or more pharmaceutically acceptable excipients to form a third mixture; and
mixing the first mixture, the second mixture, and the third mixture.

22-60. (canceled)

61. A method of manufacturing a pharmaceutical powder composition comprising at least a first type of tree nut flour, the method comprising:
roasting at least a first type of tree nut at a temperature between about 90° C. and about 140° C. to obtain a desired moisture content;
forming the at least a first type of tree nut flour using the at least a first type of tree nut after roasting;
forming the pharmaceutical powder composition by mixing the at least a first type of tree nut flour with one or more pharmaceutically acceptable excipients;
packaging the pharmaceutical powder composition.

62. The method of claim 61, wherein roasting the at least a first type of tree nut is at a temperature between about 90° C. and about 130° C.

63. The method of claim 61, wherein the pharmaceutical powder composition is packaged in a dosage form in an amount suitable for oral immunotherapy.

64. The method of claim 61, wherein the pharmaceutical powder composition comprises a second type of tree nut flour, and forming the pharmaceutical powder composition comprises mixing the at least a first type of tree nut flour, the second type of tree nut flour, and the one or more pharmaceutically acceptable excipients.

65-102. (canceled)
